(19) United States
(12) Patent Application Publication
(10) Pub. No.: US 2021/0369680 A1
(43) Pub. Date: Dec. 2, 2021

(54) METHODS OF TREATING DISEASES AND DISORDERS RESULTING FROM BETA CORONAVIRUS INFECTION

(71) Applicant: T3D Therapeutics, Inc., Research Triangle Park, NC (US)

(72) Inventors: Stanley D. CHAMBERLAIN, Wilson, WY (US); John DIDSBURY, Raleigh, NC (US)

(21) Appl. No.: 17/244,226

(22) Filed: Apr. 29, 2021

Related U.S. Application Data
(60) Provisional application No. 63/017,160, filed on Apr. 29, 2020.

(51) Int. Cl. A61K 31/421; A61K 31/675; A61K 31/167; A61K 31/4965; A61K 31/155; A61K 31/245; A61K 31/519; A61K 31/4178; A61K 39/395; A61P 31/14

(52) U.S. Cl. CPC A61K 31/421; A61K 31/675; A61K 31/167; A61K 31/4965; A61P 31/14; A61K 31/245; A61K 31/519; A61K 31/4178; A61K 39/3955; A61K 31/155

(57) ABSTRACT

This disclosure describes the use of indane acetic acid derivatives which are PPAR agonists, including PPAR delta, PPAR gamma, and dual PPAR delta and gamma agonists, and which penetrate the blood brain barrier to achieve effective brain to plasma drug levels at non-toxic doses, for the treatment of betacoronavirus diseases, such as COVID-19 and COVID-19 related co-morbid diseases, including Acute Respiratory Distress and CNS disorders, such as delirium and cognitive impairment.

### FIELD OF THE INVENTION

The present disclosure relates to the use of brain penetrating indane acetic acids and their derivatives, including mono or dual PPAR delta and gamma agonists, for the treatment of symptoms or pathological sequelae resulting from beta coronavirus infection, including for example, human coronaviruses such as SARS coronaviruses, MERS coronaviruses, and COVID-19, including Acute Respiratory Distress Syndrome (ARDS) and cognitive impairment associated with the viral infection.

### BACKGROUND OF THE INVENTION

Reports of a new coronavirus viral infection surfaced in late December 2019 in Wuhan, China. Shortly thereafter, it was reported that the virus responsible for causing the infections was contagious between humans. By early January, terms like “the new coronavirus” and “Wuhan coronavirus” were in common use. On Feb. 11, 2020, a taxonomic designation “severe acute respiratory syndrome coronavirus 2” (SARS-CoV-2) became the official means to refer to the new virus, that was previously termed as 2019-nCoV and Wuhan coronavirus. On the same day, the World Health Organization officially renamed the disease as COVID-19, which is an acronym for coronavirus disease 2019. As of mid-April, 2020, COVID-19 had affected over two million people in at least 185 countries worldwide with most of the cases being reported from China, Europe, and United States of America. Accompanying this rapid rate of infection are the absolute number of deaths, which surpassed 130,000 people globally and is expected to increase further as the disease spreads.

The complete genome of SARS-CoV-2 from Wuhan, China was submitted on Jan. 17, 2020 in the National Center for Biotechnology (NCBI) database, with ID NC_045512. The genome of SARS-CoV-2 is a 29,903 bp single-stranded RNA (ss-RNA) coronavirus. It has now been shown that the virus causing COVID-19 is a SARS-like coronavirus that had previously been reported in bats in China.

SARS-CoV-2 (COVID-19) is not the first beta coronavirus which infects humans; the pathogenic viruses that cause human diseases (human coronaviruses, HCoV) include 6 other members designated as SARS-CoV, MERS-CoV, HCoV-HKU1, HCoV-NL63, HCoV-OC43, and HCoV-229E.

Human coronaviruses were first identified in the mid-1960s. They were named for the crown-like spikes on their surface. The primary target cells for SARS-CoV-1 and SARS-CoV-2 are the epithelial cells of the respiratory and gastrointestinal tract, which contain angiotensin converting enzyme 2 (ACE2), that is utilized by the virus to enter the cell. The coronavirus SARS-CoV-2 enters human cells by binding to ACE2 via the envelope spike glycoprotein found on the surface of the virus and is also responsible for host-to-host transmission. In addition, the cellular serine protease TMPRSS2 is required to prime viral entry via ACE2. Any cell type which expresses the cellular receptor ACE2 in may potentially be infected by a beta coronavirus, such as SARS-CoV-1 and SARS-CoV-2. Other beta coronaviruses, such as MERS-CoV use different cellular receptors to enter cells.

Patients with SARS-CoV-1 or MERS present with various clinical features, ranging from asymptomatic or mild respiratory illness to fulminant severe acute respiratory disease with extrapulmonary manifestations. Both diseases have predominantly respiratory manifestations, but extrapulmonary features may occur in severe cases. Notably, early treatment is especially important for patients with severe MERS because this disease progresses to respiratory distress, renal failure and death much more rapidly than SARS-CoV-1 does. The three- to four-fold higher case-fatality rates for MERS relative to SARS-CoV-1 infection may be related to the higher median age and prevalence of comorbidities in patients with MERS as well as the different pathogenesis of the two diseases. Comorbidities associated with severe MERS include obesity, diabetes mellitus, systemic immunocompromising conditions and chronic cardiac and pulmonary diseases. Although the rate of secondary transmission among household contacts of index MERS patients (which is approximately 4%) and the estimated pandemic potential of MERS-CoV are lower than those of SARS-CoV-1, the rapidly progressive clinical course and high fatality of MERS continues to pose a major threat to at-risk populations.

The clinical presentation of infection of COVID-19 is mainly manifested as malignant pneumonia; although many patients present neurological symptoms, such as vomiting, dizziness, headache, and delirium. A current list of COVID-19 symptoms identified by the Centers of Disease Control (CDC) include: fever, cough, shortness of breath or difficulty breathing, chills, repeated shaking with chills, muscle pain, headache, sore throat, loss of taste or sense of smell, persistent pain or pressure in the chest, confusion or inability to arouse, bluish lips or face, diarrhea, or vomiting. The severity levels of COVID-19 are measured as follows:
1. Mild illness: without symptoms and signs of severe and critically infection
2. Severe illness (according to one of the following criteria):
   a. Breathing difficulties, respiratory rate≥30 bpm;
   b. SpO₂≤93% at rest;
   c. PaO₂/FiO₂≤300 mmHg.
3. Critical illness: (according to one of the following criteria):
   a. Respiratory failure, with need for mechanical ventilation assistance;
   b. Shock;
   c. Multi-organ failures, with need transport to intensive care unit (ICU).

Although the overall mortality rate of COVID-19 is low (1.4-2.3%), patients with comorbidities are more likely to have severe disease and subsequent mortality. Most available studies have shown that diabetes mellitus (DM) is associated with more severe disease, acute respiratory distress syndrome and increased mortality. Of the 32 non-survivors from a group of 52 intensive care unit (ICU) patients, DM (22%) was a predominant underlying comorbidity. Of the 1099 confirmed COVID-19 patients reported by a study from China, patients with severe disease (173) had a higher prevalence of DM (16.2%) as compared to those with non-severe disease (5.7%). Further, in a large study reported by the Chinese Center for Disease Control and Prevention comprising of 72,314 cases of COVID-19, patients with DM had higher mortality (7.3% in DM vs. 2.3% overall). See, Rimesh, 2020, COVID-19, diabetes mellitus and ACE2: The conundrum, herein incorporated by reference with regard to such background teaching.

Diabetes has been uniformly reported to be associated with poor prognosis in other viral infections, notably seasonal influenza, pandemic influenza A H1N1 (2009), Severe Acute Respiratory Syndrome (SARS) and Middle East Respiratory Syndrome (MERS). Multiple possible explanations exist for the association between pre-existing DM and COVID-19 severity. Innate immunity, the first line of defense against SARS-CoV-2, is compromised in patients with uncontrolled DM thereby allowing proliferation of the pathogen within the host. Short-term hyperglycemia has been shown to slow the innate immune system. Moreover, DM is characterized by exaggerated pro-inflammatory cytokine response, notably interleukin (IL)-1, IL-6 and tumor-necrosis factor (TNF)-α, which may be further exaggerated in in patients with COVID-19 complicated by acute respiratory distress syndrome (ARDS).

The virus receptor, angiotensin-converting enzyme 2 (ACE2), may be involved in the association between DM and COVID-19. ACE2 is a type 1 integral membrane glycoprotein that is constitutively expressed by the epithelial cells of the lungs, kidney, intestine and blood vessels. In normal physiology, ACE2 breaks down angiotensin-II and to a lesser extent, angiotensin-I to smaller peptides, angiotensin (1-7) and angiotensin (1-9), respectively. The ACE2/Ang (1-7) system plays important anti-inflammatory and anti-oxidant roles to protect the lung against ARDS. ACE2 expression is reduced in patients with DM, which might explain the increased predisposition to severe lung injury and ARDS with COVID-19. See, Bornstein, 2020, Endocrine and metabolic link to coronavirus infection, herein incorporated by reference with regard to such background teaching. Angiotensin 1-7 acts on the Mas receptor pathway, which leads to anti-inflammatory and anti-fibrotic responses that might aid the recovery of patients with a beta coronavirus infection such as COVID-19. It could be postulated that individuals with more severe COVID-19 have an imbalance in the Renin Angiotensin System (RAS), with a decrease in Angiotensin 1-7 and an increase in Angiotensin 2 resulting in hypertension and insulin-resistance.

A possible explanation for a link between hyperglycemia and ACE2 levels in the severity of COVID-19 disease could be explained by several clinical observations in SARS and preclinical observations in the NOD diabetic mouse. Changes in glycosylation of the ACE2, as well as glycosylation of the viral spike protein, both possibly induced by uncontrolled hyperglycemia, may alter both the binding of the viral spike protein to ACE2 and the degree of the immune response to the virus. See, Brufsky 2020, Hyperglycemia, hydroxychloroquine, and the COVID-19 pandemic, herein incorporated by reference with regard to such background teaching.

Studies have shown that SARS-CoV may damage the kidney, heart, lung, and the endocrine part of the pancreas as indicated by assays for the biomarkers s-Cr, LDH, CKMB, SaO2, and FPG and that these biomarkers were predictors of mortality and correlated with ACE2 expression. ACE2 expression in the exocrine and endocrine tissues of the pancreas suggests that beta coronaviruses such as SARS-CoV-1 and -2 may damage pancreatic islets and cause acute insulin dependent diabetes mellitus. See, Yang, 2010, Binding of SARS coronavirus to its receptor damages islets and causes acute diabetes, herein incorporated by reference with regard to such background teaching.

In reports of the clinical characteristics of patients with COVID-19 infection, hyperglycemia was noted in 51% of cases. Transient hyperglycemia was also noted in patients with SARS in 2003, caused by SARS-CoV-1. The SARS-Cov-1 virus leads to transient impairment of pancreatic islet cell function. The closely related, Middle Eastern Respiratory Syndrome (MERS in 2013) coronavirus (MERS-CoV) as well as human coronavirus-EMC are anchored to host cells via dipeptidyl peptidase 4 (DPP-4), which physiologically is implicated in the modulation of insulin action and glucose metabolism and is responsible for the degradation of incretins such as glucagon like peptide-1, (GLP-1). See, Ioannis Ilias 2020, Hyperglycemia and the novel Covid-19 infection: Possible pathophysiologic mechanisms, herein incorporated by reference with regard to such background teaching.

The pathogenesis of severe acute respiratory syndrome (SARS) related to beta coronavirus infection involves a so-called ‘cytokine storm,’ with high serum levels of pro-inflammatory cytokines and chemokines interleukin 6 (IL6), tumor necrosis factor (TNF-alpha), interferon-gamma, IL-1 and IL-12, and IL-8. Similarly, in COVID-19, higher plasma levels of cytokines IL-6, IL-2, IL-7, IL-10, interferon gamma inducible protein (IP10), monocyte chemo attractant protein (MCP1), macrophage inflammatory protein (MIP1A) and TNF-alpha have been found in patients admitted to intensive care units, and the degree of the cytokine storm was proportional to disease severity. The pro-inflammatory cytokine IL-6 has a prominent role in the inflammatory cascade, and may result in increased alveolar-capillary blood-gas exchange dysfunction. Modulation of IL-6 may be a potential target to treat COVID-19-related ARDS. In a case study of a patient with a respiratory failure linked to COVID-19, the patient had a rapid favorable outcome after two infusions of an anti-interleukin 6 receptor inhibitor. This suggests that anti-IL6 receptor inhibitor treatment could decrease the risk of progression toward SARS by mitigating the cytokine storm in the lungs with COVID-19. See, Michot 2020, Tocilizumab, an anti-IL6 receptor antibody, to treat Covid-19-related respiratory failure: a case report, herein incorporated by reference with regard to such background teaching.

Evidence suggests that a subgroup of patients with severe COVID-19 could have a dysregulation of the immune response that allows the development of viral hyperinflammation. Patients with severe COVID-19 may be screened for hyperinflammation using standard laboratory parameters. Neutrophil-to-lymphocyte ratio (NLR) and lymphocyte-to-C-reactive protein ratio (LCR) are established inflammation markers that reflect systemic inflammatory response. In a meta analysis of studies that included a total number of 828 patients, where 407 (49.15%), patients had severe disease, the NLR values were found to increase significantly in patients with COVID-19 with severe disease (SMD=2.404, 95% CI=0.98-3.8 2), while LCR values were decreased significantly (SMD=−0.912, 95% CI=−1.275 to −0.5 50). Several other reports describe increased levels of neutrophils and C-reactive protein along with a decrease in lymphocyte numbers in patients with COVID-19. ARDS, characterized by a rapid onset of generalized inflammation in the lungs, is the leading cause of mortality of patients with COVID-19. Thus, increased NLR levels and low LCR levels reflecting an enhanced inflammatory process may suggest a poor prognosis. See, Francisco 2020, Neutrophil-to-lymphocyte ratio and lymphocyte-to-C-reactive protein ratio in patients with severe coronavirus disease 2019 (COVID-19): A meta-analysis, herein incorporated by reference with regard to such background teaching.

It has been shown that inflammasome NLRP3 is a major pathophysiological component in the development of acute respiratory distress syndrome (ARDS), while structural models have shown that SARS-CoV-2 proteins such as viroporins E, 3a, and 8A play a substantial role in viral replication and pathogenic sequelae, and that these three proteins provoke the activation of inflammasome NLRP3. See, Deftereos 2020, The Greek study in the effects of colchicine in Covid-19 complications prevention (GRECCO-19 study): Rationale and study design, herein incorporated by reference with regard to such background teaching.

The ORF3a protein expressed by SARS-CoV-2 has 72% sequence homology with SARS-CoV ORF3a. In SARS-CoV, the ORF3a protein activates NF-κB and the NLRP3 inflammasome by inducing TRAF3-dependent ubiquitination of p105 and ASC. See, Kam-Leung Siu, 2019, Severe acute respiratory syndrome coronavirus ORF3a protein activates the NLRP3 inflammasome by promoting TRAF3-dependent ubiquitination of ASC, herein incorporated by reference with regard to such background teaching. There is evidence that the SARS-CoV-2 virus is less effective in activating the NLRP3 inflammasome and in suppressing the antiviral response when compared to SARS-CoV. More studies are needed to fully understand how the SARS-CoV-2 ORF3a protein influences the immune and inflammatory response as it pertains to COVID-19 disease progression.

Multiple studies have shown that PPAR-γ agonists inhibit the activation of inflammasome NLRP3. See, Si Ming Li 2019, PPAR-γ Activation Exerts an Anti-inflammatory Effect by Suppressing the NLRP3 Inflammasome in Spinal Cord-Derived Neurons, herein incorporated by reference with regard to such background teaching. Studies in spinal chord injury (SCI) models suggest that PPAR activation exerts an anti-inflammatory effect by suppressing the NLRP3 inflammasome in neurons.

Clinical and preclinical data from studies with coronaviruses suggest wider tissue invasiveness and neurotropism, which may result in more complex clinical disease. It has been demonstrated that coronavirus infection, and especially beta coronavirus infection, is not limited to the respiratory tract and frequently affect the central nervous system (CNS). This neurotropism has been documented for the SARS-CoV-1, MERS-CoV and the coronavirus responsible for porcine hemagglutinating encephalomyelitis (HEV 67N) 3-5. The ACE2 receptor for SARS-CoV-2 is expressed in the brain, especially in the brain stem and in regions responsible for regulation of cardiovascular function including subfornical organ, paraventricular nucleus (PVN), nucleus of the tractus solitarius (NTS), and rostral ventrolateral medulla; expression of ACE2 was found in both neurons and glia. Non-ACE2 pathways for virus infection of neural cells cannot be excluded; the significant penetration of betacoronavirus into liver, an organ with lower levels of ACE2 compared to the CNS, supports the assumption that cell entry routes for betacoronaviruses may vary. CNS infection with both SARS-CoV-1, MERS-CoV have been reported, and SARS-CoV-1 has been identified in neurons from tissues obtained from infected patients. Since SARS-CoV-2 also enters cells through the ACE2 receptor, it is very likely it also infects neural cells, and CNS damage is a component pathophysiology of COVID-19. See, Steardo 2020, Neuroinfection may contribute to pathophysiology and clinical manifestations of COVID-19, herein incorporated by reference with regard to such background teaching.

Betacoronaviruses may enter the CNS through several routes, most notably through intranasal inoculation and though peripheral nerves using trans-synaptic pathways. Betacoronaviruses may infect both neurons and neuroglia; neural cells express the entry protein ACE2, although direct endocytotic infection (similar to those demonstrated for ZIKA and TBEV viruses) cannot be excluded. Direct CNS infection together with systemic inflammation, which accompanies COVID-19, compromises the blood brain barrier and triggers a massive neuroinflammatory response manifested by reactive astrogliosis and activation of microglia. Neuroinflammation together with prolonged hypoxia may promote neuropsychiatric developments and cognitive impairments, both acute and chronic. The neurological and psychiatric aspects of the viral attack must therefore be considered in designing therapeutic strategies and rehabilitation programs for SARS-CoV-2 infected human subjects. See, Steardo 2020, Neuroinfection may contribute to pathophysiology and clinical manifestations of COVID-19, herein incorporated by reference with regard to such background teaching.

A recent study involving 214 COVID-19 patients, reported neurological manifestations in 78 (36.4%) of the patients, supporting the neurotropic potential in the COVID-19 virus (SARS-CoV-2). See, Mao 2020, Neurologic Manifestations of Hospitalized Patients With Coronavirus Disease 2019 in Wuhan, China, herein incorporated by reference with regard to such background teaching.

Patients neurological symptoms included headache, disturbed consciousness, and paresthesia. In addition, severely affected patients were more likely to develop neurological symptoms than patients who had mild or moderate disease. Autopsy reports have revealed brain tissue edema and partial neuronal degeneration in deceased patients. At least one case of viral encephalitis caused by SARS-CoV-2 has been reported and SARS-CoV-2 was detected in cerebrospinal fluid. This study illustrates that COVID-19 has the potential to cause nervous system damage. See, Wu 2020, Nervous system involvement after infection with COVID-19 and other coronaviruses, herein incorporated by reference with regard to such background teaching.

In a retrospective analysis of 4189 confirmed COVID-19-infected patients from 28 studies more severe COVID-19 infection was associated with higher mean troponin (SMD 0.53, 95% CI 0.30 to 0.75, p<0.001), with a similar trend for creatine kinase-MB, myoglobin, and NT-proBNP. Acute cardiac injury was more frequent in those with severe, compared to milder, disease (risk ratio 5.99, 3.04 to 11.80; p<0.001). Meta regression suggested that cardiac injury biomarker indicators useful for differentiating severity are related to a history of hypertension (p=0.030). COVID19-related cardiac injury is associated with higher mortality (summary risk ratio 3.85, 2.13 to 6.96; p<0.001). hsTnl and NT-proBNP levels increased during the course of hospitalization only in non-survivors. Severity of COVID-19 is associated with acute cardiac injury, and acute cardiac injury with death. Cardiac injury biomarkers increased predominantly in non-survivors. See, Li J W, 2020, The impact of 2019 novel coronavirus on heart injury: A systemic review and Meta-analysis, herein incorporated by reference with regard to such background teaching.

Hospitalized, bedridden patients are particularly prone to develop deep venous thrombosis (DVT), which has an overall incidence among in-hospital patients of about 0.9%, and up to 15-32% among intensive care unit (ICU) patients. Among COVID-19 infected patients, there have been a significant increase in the diagnoses of DVT among both ICU and non-ICU hospitalized patients. DVT may be considered as a frequent and potentially lethal complication of COVID-19, which deserves further attention in order to establish incidence, mortality rate, and the opportunity of a screening program and prophylactic therapy. See, Marone 2020, Upsurge of deep venous thrombosis in patients affected by COVID-19: Preliminary data and possible explanations, herein incorporated by reference with regard to such background teaching.

Increased rates of liver dysfunction have been observed in patients with severe COVID-19, regardless of preexisting conditions. Patients suffering from non-alcoholic fatty liver disease (NAFLD) often present with elevated cytokine levels, these patients may also be more vulnerable to increased cytokine production associated with COVID-19. Thus NAFLD progression could be enhanced by COVID-19. Patients with NAFLD may be especially vulnerable to SARS-CoV-2 infection and its complications, SARS-CoV-2 infection may also increase NAFLD progression to non-alcoholic steatohepatitis (NASH). These observations underline the importance of identification and monitoring of patients with pre-existing liver disease, especially those with metabolic disorder, in the COVID-19 pandemic and utilizing prophylactic therapy. See, Prins 2020, Potential implications of COVID-19 in non-alcoholic fatty liver disease, herein incorporated by reference with regard to such background teaching.

In addition to the alveolar cells in the lungs, ACE2 is expressed in other organs, including the kidney, heart and GI tract. Whether robust ACE2 expression in these organs affects SARS-CoV-2 infectivity remains ill-defined, but the finding that acute kidney injury (AKI), cardiac damage and abdominal pain are the most commonly reported co-morbidities of COVID-19 suggests that SARS CoV-2 may have a tropism for these organs. However, whether SARS-CoV-2 replication actually occurs in these organs, possibly affecting their functional homeostasis and contributing to the virus spreading throughout the body is unknown. It has been reported that in the kidney, ACE2 is highly expressed in the brush border of proximal tubular cells and, to a lesser extent, in podocytes, but not in glomerular endothelial and mesangial cells. In earlier studies during the SARS outbreak in 2003, it was found that 6% of SARS-CoV-infected subjects experienced AKI. See, Perico 2020, Should COVID-19 Concern Nephrologists? Why and to What Extent? The Emerging Impasse of Angiotensin Blockade, herein incorporated by reference with regard to such background teaching.

Cytokine Release Syndrome (CRS), or Cytokine Storm, is a systemic inflammatory response, which may be caused by infection, certain drugs and other factors, it is characterized by a supranormal increase in the level of a large number of pro-inflammatory cytokines. CRS is most common in immune system-related diseases or immune-related therapy, such as CAR-T cell therapy, organ transplantation sepsis, and virus infection. The SARS-CoV-2 binds to alveolar epithelial cells, then the virus activates the innate immune system and the adaptive immune system, resulting in the release of a large number of cytokines, including IL-6. These pro-inflammatory factors, increase vascular permeability, to increase fluid and cell infiltration into alveoli, resulting in dyspnea and respiratory failure. It is reasonable to hypothesize that cytokine storms in severe COVID-19 cases are a major cause of respiratory failure, therefore, neutralizing or reducing the levels of key inflammatory factors in CRS may be of value in reducing mortality. See, Zhang 2020, The cytokine release syndrome (CRS) of severe COVID-19 and Interleukin-6 receptor (IL-6R) antagonist Tocilizumab may be the key to reduce the mortality, herein incorporated by reference with regard to such background teaching.

Indane acetic acid PPAR agonists detailed herein were disclosed in Lowe et al., US 2005/0075338, and related U.S. Pat. No. 6,828,335. U.S. Pat. No. 6,828,335 is hereby incorporated by reference in its entirety. The present disclosure describes new uses for the compounds described in U.S. Pat. No. 6,828,335.

PPARs (peroxisome proliferator-activated receptors, are a family of ligand-activated transcription factors that play an essential role in cellular processes such as cell differentiation, inflammation, and metabolism. The PPARs are ligand-activated transcription factors belonging to the nuclear hormone receptor superfamily, and exist as three different isoforms: PPARα, PPARδ (also called β), and PPARγ. PPARs are activated by lipids and fatty acid derivatives, and they carry out essential functions in lipid homeostasis, glucose metabolism, energy production and cellular differentiation. PPARs are expressed through out the body in a variety of cell types including microglia, astrocytes, oligodendrocytes and neurons. Compounds which have individual, or single, PPARα, PPARδ, or PPARγ agonist activity are thought to have some potential as systemic therapeutics. Dual or triple PPAR isoform agonists are not well studied and their potential as therapeutics is not well understood. Only recently have there been discovered compositions with dual PPARδ and PPARγ agonist activity where PPARδ activity is greater than PPARγ and PPARα activity.

An indane acetic acid PPAR delta agonist of the present disclosure has been used in a clinical study in elderly Alzheimer's disease subjects (age range of 50 to 85) dosed daily for two weeks. Systemic measurements of a large array of metabolic markers (metabolomics) demonstrated dose-dependent marker changes that are correlative to anti-diabetic effects, including lowering levels of branched chain amino acids. In the same study a dose-dependent decrease in fasting plasma glucose was observed, further supporting anti-diabetic activity of the agonist. In a previous Phase 1 study, adiponectin levels were increased in a dose dependent fashion over the period of one week. Adiponectin is an insulin-sensitizing hormone released from fat cells.

The time course of COVID-19 infection in many patients and the time course observed for a pharmacological response from an indane acetic acid of the present disclosure are similar.

For a therapeutic to be effective in treating CNS disease such as cognitive impairment resulting from COVID-19 infection, the therapeutic must be able pass from blood through the blood-brain barrier (BBB) and into the brain extracellular fluid (BECF) in the central nervous system (CNS). The blood-brain barrier is formed by endothelial cells, which are connected by tight cell junctions. The blood-brain barrier allows the passage of water, gases, and lipid-soluble molecules by passive diffusion, as well as the selective transport of molecules such as glucose and amino acids that are crucial to neural function. The blood-brain barrier also eliminates lipophilic molecules by way of an active transport mechanism mediated by P-glycoprotein (P-gp), or other efflux transporters such as Organic anion transporter 3 (Oat3) and the peptide transporter 2 (PEPT2). For a cognitive therapeutic to be effective, it must achieve balance between passive diffusion through the BBB, and active elimination out of the brain by the P-gp transporter, or other transporters. P-gp is an ATP-dependent, drug efflux pump for xenobiotic compounds with broad tissue distribution including the endothelia cells of the BBB. One measure of whether a small molecule penetrates the BBB and is not rapidly transported out, is the brain to plasma ratio of the drug. This may be measured in pre-clinical animal models by determining plasma concentration vs time curves as in a standard pharmacokinetic study, and in addition harvesting brains and determining whole brain concentrations over time. The brain to plasma concentration ratio may them be determined at any time point, such as the Cₘₐₓ, or for the entire time curve (AUC, area under the curve). Alternatively, brain exposure may be determined by measuring drug concentrations in ventricular and lumbar CSF. Clinically, brain FDG-PET may be used as an indirect measure of the pharmacological effect of a therapeutic and of brain levels of drug. DSST is among the most commonly used tests in clinical neuropsychology, and has been used to measure a range of cognitive functions including intact motor speed, attention, executive, and visuo-perceptual functions. DSST has been used to test the effect of antidepressants such as Vortioxetine. The DSST test is a paper-and-pencil cognitive measure performed on a simple form that asks a subject to match symbols in a chart to a key at the top of the page. The number of correct symbols charted within two minutes constitutes the score, which may range from zero to 133. Brevity, reliability, and language and education independence make DSST a very commonly used test in neuropsychology.

There is a need for treatments for betacoronovirus infections, including treatments for the diseases and disorders created or exacerbated by the viral infection.

### SUMMARY OF THE INVENTION

One embodiment of the present disclosure includes a method for treating a subject having a beta coronavirus infection comprising administering a therapeutically effective amount of a Peroxisome Proliferator-Activated Receptor (PPAR) agonist, which penetrates the blood brain barrier (BBB).

One aspect of the present disclosure includes wherein the beta coronovirus is selected from one or more of SARS-CoV-2, SARS-CoV-1, MERS-CoV, NCoV-0043, HCoV-HKU1, and a novel beta coronavirus.

One aspect of the present disclosure includes wherein the beta coronavirus infection causes or exacerbates one or more of Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), and elevated blood glucose levels.

One aspect of the present disclosure includes wherein the therapeutically effective amount provides pharmacologically useful concentrations in the brain.

One aspect of the present disclosure includes wherein the PPAR agonist is a PPAR-delta agonist, a PPAR-gamma agonist, or a dual PPAR delta and gamma agonist.

One aspect of the present disclosure includes wherein the PPAR agonist is a compound of Formula I:

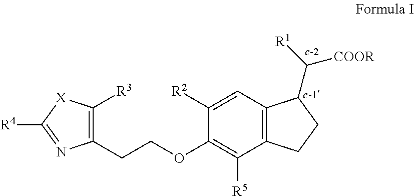

R is H or C₁-C₆ alkyl;
R¹ is H, COOR, C₃-C₈ cycloalkyl, or C₁-C₆ alkyl, C₂-C₆ alkenyl, or C₁-C₆ alkoxy, each of which may be unsubstituted or substituted with fluoro, methylenedioxyphenyl, or phenyl which may be unsubstituted or substituted with R⁶;

### R² is

(i) H, halo, or C₁-C₆ alkyl, which may be unsubstituted or substituted with C₁-C₆ alkoxy, oxo, or fluoro; or
(ii) phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl, each of which may be unsubstituted or substituted with R⁶;
   R³ is H, C₁-C₆ alkyl, or phenyl which may be unsubstituted or substituted with R⁶;

### X is O or S;

### R⁴ is

(i) C₁-C₆ alkyl or C₃-C₈ cycloalkyl,
   a. either of which may be unsubstituted or substituted with fluoro, oxo, or C₁-C₆ alkoxy, which may be unsubstituted or substituted with C₁-C₆ alkoxy or phenyl optionally substituted with R⁶,
      or
   b. either of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1, 4-benzodioxanyl, each of which may be unsubstituted or substituted with R⁶,
      or
   c. C₁-C₆ alkyl may also be substituted with
      i. C₃-C₈ cycloalkyl;
      ii. phenoxy which may be unsubstituted or substituted with R⁶; or
      iii. phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1, 4-benzodioxanyl, each of which may be unsubstituted or substituted with R⁶, or
(ii) phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl, each of which may be unsubstituted or substituted with R⁶ or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy, each of which may be unsubstituted or substituted with R⁶;
   R⁵ is H, halo, or C₁-C₆ alkyl optionally substituted with oxo;
   R⁶ is halo, CF₃, C₁-C₆ alkyl optionally substituted with oxo or hydroxy, or C₁-C₆ alkoxy optionally substituted with fluoro;
   or a pharmaceutically acceptable salt or ester thereof.

One aspect of the present disclosure includes wherein
R¹ is H or C₁-C₆ alkyl;
R² is H or halo;
R³ is C₁-C₆ alkyl;
R⁴ is unsubstituted phenyl or phenyl substituted with one or more halogen, C₁-C₆ alkyl, C₁-C₆ haloalkyl, or C₁-C₆ alkoxy;
R⁵ is H or halo; and
X is O or S.

One aspect of the present disclosure includes wherein the designated c-1′ has S relative stereochemistry.

One aspect of the present disclosure includes wherein the PPAR agonist is selected from:

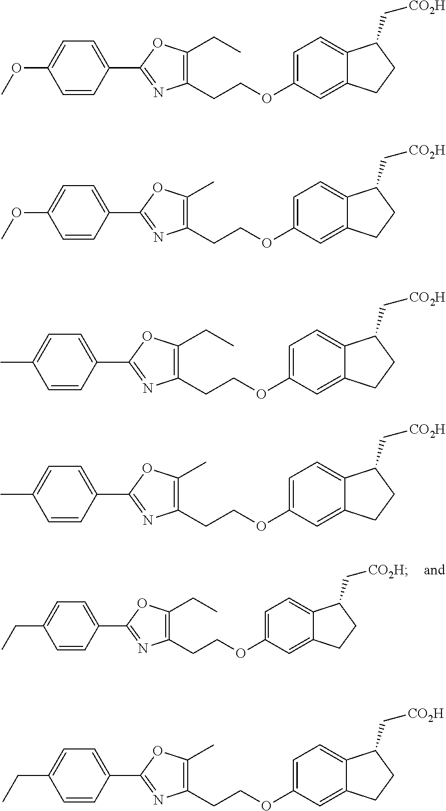 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

or a pharmaceutically acceptable salt or ester thereof.

One aspect of the present disclosure includes wherein the pharmaceutically acceptable salt is selected from the group consisting of alkali metal salts, alkaline earth metal salts, ammonium salts with organic bases, and basic nitrogen containing groups in the conjugate base that is quaternized with agents selected from the group consisting of alkyl halides and aralkyl halides, or other alkylating agents.

One aspect of the present disclosure includes wherein salt is a potassium, sodium, calcium, magnesium, lysine, choline or meglumine salt thereof.

One aspect of the present disclosure includes wherein the PPAR agonist is (1S)-1H-Indene-1-acetic acid, 5-[2-[5-ethyl-2-(4-methoxyphenyl)-4-oxazolyl]ethoxy]-2,3-dihydro-, sodium salt (1:1).

One aspect of the present disclosure includes wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat Acute Respiratory Distress Syndrome (ARDS) associated with COVID-19 disease.

One aspect of the present disclosure includes wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat a central nervous system disorder, delirium, or cognitive impairment associated with COVID-19 disease.

One aspect of the present disclosure includes wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat cardiovascular disease associated with COVID-19 disease.

One aspect of the present disclosure includes wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat kidney disease associated with COVID-19 disease.

One aspect of the present disclosure includes wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat intestinal disease associated with COVID-19 disease.

One aspect of the present disclosure includes wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat liver disease associated with COVID-19 disease.

One aspect of the present disclosure includes wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat deep vein thrombosis associated with COVID-19 disease.

One aspect of the present disclosure includes wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat elevated blood glucose levels associated with COVID-19 disease.

One aspect of the present disclosure includes wherein the PPAR agonist is administered intravenously, orally, buccally, transdermally, rectally, nasally, optically, intrathecally, or intra-cranially

One embodiment of the present disclosure further includes administration of one or more additional therapeutic agents. One aspect of the present disclosure includes wherein one or more additional therapeutic agent is used to treat COVID-19. One aspect of the present disclosure includes wherein the one or more additional therapeutic agent is an antiviral.

One aspect of the present disclosure includes wherein one or more additional therapeutic agents is chloroquine, hydroxychloroquine, remdesivir or nafamostat mesylate.

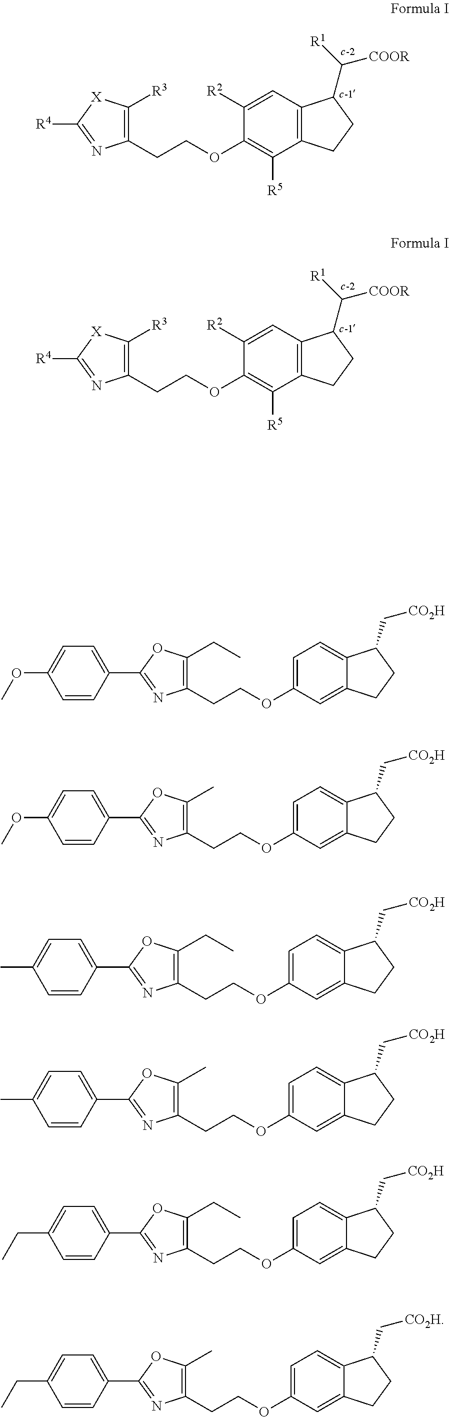

In another embodiment, the indane acetic acid used to treat a beta coronavirus infection, and related CNS disease, is selected from the list of six above and has a rat brain to plasma ratio of greater than 20% 12 hours after oral dosing.

The present disclosure includes the various embodiments and aspects in combination as if such were explicitly recited.

### DETAILED DESCRIPTION OF THE INVENTION

### Definitions

The term “halo” means F, Cl, Br, or I.

The term “C₁-C₆ alkyl” means a straight or branched saturated hydrocarbon carbon chain of from 1 to 6 carbon atoms, respectively. Examples of such groups include methyl, ethyl, isopropyl, sec-butyl, 2-methylpentyl, n-hexyl, and the like.

The term “C₂-C₆ alkenyl” means a straight or branched unsaturated hydrocarbon carbon chain of from 2 to 6 carbon atoms. Examples of such groups include vinyl, allyl, isopropenyl, 2-butenyl, 3-ethyl-2-butenyl, 4-hexenyl, and the like.

The term “C₁-C₆ haloalkyl” means a C₁-C₆ alkyl group substituted by halogen atoms up to the perhalo level. Examples of such groups include trifluoromethyl, tetrafluoroethyl, 1,2-dichloropropyl, 6-iodohexyl, and the like.

The terms “C₃-C₆ cycloalkyl” and “C₃-C₈ cycloalkyl” mean a saturated carbocyclic ring system of from 3 to 6 carbon atoms or from 3 to 8 carbon atoms, respectively. Examples of such groups include cyclopropyl, cyclobutyl, cyclopentyl, cyclohexyl, and the like.

The term “C₁-C₆ acyl” means a C₁-C₆ alkyl group attached at the carbonyl carbon atom. The radical is attached to the rest of the molecule at the carbonyl bearing carbon atom. Examples of such groups include acetyl, propionyl, n-butanoyl, 2-methylpentantoyl, and the like.

The term “C₁-C₆ alkoxy” means a linear or branched saturated carbon group having from 1 to 6 carbon atoms, said carbon group being attached to an O atom. The O atom is the point of attachment of the alkoxy substituent to the rest of the molecule. Such groups include, but are not limited to, methoxy, ethoxy, n-propoxy, isopropoxy, and the like.

The term “C₁-C₆ thioalkyl” means a linear or branched saturated carbon group having from 1 to about 6 carbon atoms, said carbon group being attached to an S atom. The S atom is the point of attachment of the thioalkyl substituent to the rest of the molecule. Such groups include, for example, methylthio, propylthio, hexylthio, and the like.

The term “C₁-C₆ haloalkoxy” means a C₁-C₆ alkoxy group further substituted on C with 1 to 3 halogen atoms or fluorine up to the perfluoro level.

The term “C₃-C₈ cycloalkoxy” means a C₃-C₈ cycloalkyl group attached to an O atom. The O atom is the point of attachment of the cycloalkoxy group with the rest of the molecule.

The term “phenoxy” means a phenyl group attached to an O atom. The O atom is the point of attachment of the phenoxy group to the rest of the molecule.

The term “6-membered heteroaryl ring” means a 6-membered monocyclic heteroaromatic ring radical containing 1-5 carbon atoms and up to the indicated number of N atoms. Examples of 6-membered heteroaryl rings are pyridyl, pyrimidyl, pyridazinyl, pyrazinyl, triazinyl, and the like.

The term “5- or 6-membered heterocyclic ring” means a 5- or 6-membered ring containing 1-5 C atoms and up to the indicated number of N, O, and S atoms, and may be aromatic, partially saturated, or fully saturated.

The term “optionally substituted” means that, unless indicated otherwise, the moiety so modified may have from one to up to the number of the substituents indicated, provided the resulting substitution is chemically feasible as recognized in the art. Each substituent may replace any H atom on the moiety so modified as long as the replacement is chemically possible and chemically stable. For example, a chemically unstable compound would be one where each of two substituents is bonded to a single C atom through each substituents heteroatom. Another example of a chemically unstable compound would be one where an alkoxy group is bonded to the unsaturated carbon of an alkene to form an enol ether. When there are two or more substituents on any moiety, each substituent is chosen independently of the other substituent so that, accordingly, the substituents may be the same or different.

When the 5- or 6-membered heterocyclic ring is attached to the rest of the molecule as a substituent, it becomes a radical. Examples of 5- or 6-membered heteroaryl ring radicals are furyl, pyrrolyl, thienyl, pyrazolyl, isoxazolyl, imidazolyl, oxazolyl, thiazolyl, isothiazolyl, triazolyl, thiadiazolyl, oxadiazolyl, pyridyl, pyrimidyl, pyridazinyl, pyrazinyl, triazinyl, and the like. Examples of partially unsaturated 5- or 6-membered heterocyclic ring radicals include dihydropyrano, pyrrolinyl, pyrazolinyl, imidazolinyl, dihydrofuryl, and the like. Examples of saturated 5- or 6-membered heterocyclic ring radicals include pyrrolidinyl, tetrahydropyridyl, piperidinyl, morpholinyl, tetrahydrofuryl, tetrahydrothienyl, piperazinyl, and the like. The point of attachment of the radical may be from any available C or N atom of the ring to the rest of the molecule. When the 5- or 6-membered heterocyclic ring is fused to another ring contained in the rest of the molecule, it forms a bicyclic ring. Examples of such 5- and 6-heterocyclic fused rings include pyrrolo, furo, pyrido, piperido, thieno, and the like. The point of fusion is at any available face of the heterocyclic ring and parent molecule.

The term “subject”, as used herein, means a mammalian subject (e.g., dog, cat, horse, cow, sheep, goat, monkey, etc.), and particularly human subjects (including both male and female subjects, and including neonatal, infant, juvenile, adolescent, adult and geriatric subjects.

As used herein, “treatment”, “treat”, and “treating” refer to reversing, alleviating, mitigating, or slowing the progression of, or inhibiting the progress of, a disorder or disease as described herein.

As used herein, “an effective amount” refers to an amount that causes relief of symptoms of a disorder or disease as noted through clinical testing and evaluation, patient observation, and/or the like. An “effective amount” may further designate a dose that causes a detectable change in biological or chemical activity. The detectable changes may be detected and/or further quantified by one skilled in the art for the relevant mechanism or process. Moreover, an “effective amount” may designate an amount that maintains a desired physiological state, i.e., reduces or prevents significant decline and/or promotes improvement in the condition of interest. An “effective amount” may further refer to a therapeutically effective amount.

### Compounds

The present invention encompasses the compounds of Formula I. The compounds are believed to be PPAR delta and gamma dual agonists. Compounds of Formula (I) are as disclosed in U.S. Pat. No. 6,828,335, which is herein incorporated by reference in its entirety. The compounds of Formula (I):

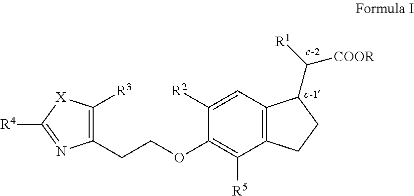

R is H or C₁-C₆ alkyl;
R¹ is H, COOR, C₃-C₈ cycloalkyl, or C₁-C₆ alkyl, C₂-C₆ alkenyl, or C₁-C₆ alkoxy, each of which may be unsubstituted or substituted with fluoro, methylenedioxyphenyl, or phenyl which may be unsubstituted or substituted with R⁶;

### R² is

(iii) H, halo, or C₁-C₆ alkyl, which may be unsubstituted or substituted with C₁-C₆ alkoxy, oxo, or fluoro; or
(iv) phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl, each of which may be unsubstituted or substituted with R⁶;
   R³ is H, C₁-C₆ alkyl, or phenyl which may be unsubstituted or substituted with R⁶;

### X is O or S;

### R⁴ is

(iii) C₁-C₆ alkyl or C₃-C₈ cycloalkyl,
   a. either of which may be unsubstituted or substituted with fluoro, oxo, or C₁-C₆ alkoxy, which may be unsubstituted or substituted with C₁-C₆ alkoxy or phenyl optionally substituted with R⁶,
      or
   b. either of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1, 4-benzodioxanyl, each of which may be unsubstituted or substituted with R⁶,
      or
   c. C₁-C₆ alkyl may also be substituted with
      i. C₃-C₈ cycloalkyl;
      ii. phenoxy which may be unsubstituted or substituted with R⁶; or
      iii. phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1, 4-benzodioxanyl, each of which may be unsubstituted or substituted with R⁶, or
(iv) phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl, each of which may be unsubstituted or substituted with R⁶ or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy, each of which may be unsubstituted or substituted with R⁶;
   R⁵ is H, halo, or C₁-C₆ alkyl optionally substituted with oxo;
   R⁶ is halo, CF₃, C₁-C₆ alkyl optionally substituted with oxo or hydroxy, or C₁-C₆ alkoxy optionally substituted with fluoro;
   or a pharmaceutically acceptable salt or ester thereof.

One aspect of the present disclosure includes wherein
R¹ is H or C₁-C₆ alkyl;
R² is H or halo;
R³ is C₁-C₆ alkyl;
R⁴ is unsubstituted phenyl or phenyl substituted with one or more halogen, C₁-C₆ alkyl, C₁-C₆ haloalkyl, or C₁-C₆ alkoxy;
R⁵ is H or halo; and
X is O or S.

One aspect of the present disclosure includes wherein the designated c-1′ has S relative stereochemistry.

One aspect of the present disclosure includes wherein the compound is selected from:

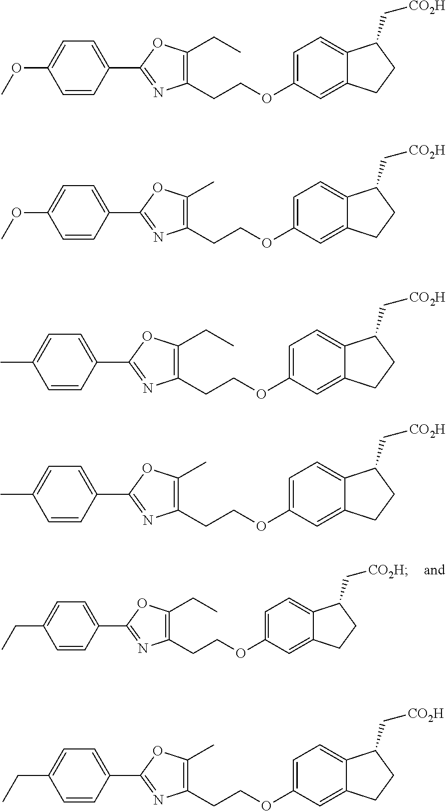 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

or a pharmaceutically acceptable salt or ester thereof.

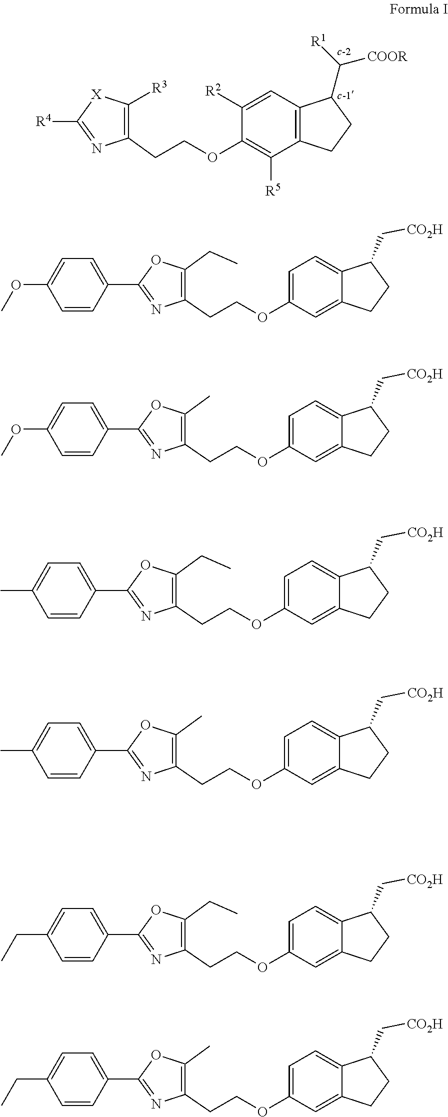

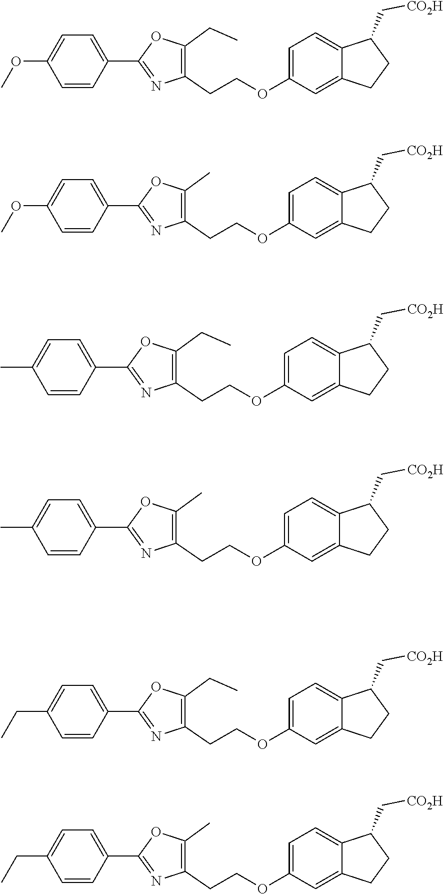 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

Exemplary compounds of Formula I are listed in Table 1 as the free acid, but may also be a pharmaceutically acceptable salt or ester thereof.

TABLE 1
Illustrative Examples of Compounds of Formula I
Formula I

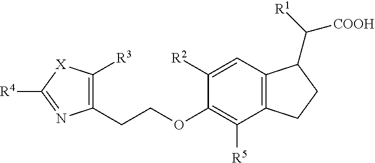

| Entry | | | | | | |
|---|---|---|---|---|---|---|
| No. | R¹ | R² | R³ | R⁴ | R⁵ | X |
| 1 | H | H | Me | Ph | H | O |
| 2 | H | H | Me | 2-F—Ph | H | O |
| 3 | H | H | Me | 2-Cl Ph | H | O |
| 4 | H | H | Me | 2-Me Ph | H | O |
| 5 | H | H | Me | 3-F—Ph | H | O |
| 6 | H | H | Me | 3-Cl Ph | H | O |
| 7 | H | H | Me | 3-CF₃ Ph | H | O |
| 8 | H | H | Me | 3-Me Ph | H | O |
| 9 | H | H | Me | 3-MeO Ph | H | O |
| 10 | H | H | Me | 4-F—Ph | H | O |
| 11 | H | H | Me | 4-Cl Ph | H | O |
| 12 | H | H | Me | 4-CF₃ Ph | H | O |
| 13 | H | H | Me | 4-Me Ph | H | O |
| 14 | H | H | Me | 4-Et Ph | H | O |
| 15 | H | H | Me | 4-MeO Ph | H | O |
| 16 | H | H | Me | 4-EtO Ph | H | O |
| 17 | H | H | Me | 2,3-di-F Ph | H | O |
| 18 | H | H | Me | 2,4-di-F Ph | H | O |
| 19 | H | H | Me | 3,4-di-F Ph | H | O |
| 20 | H | H | Me | 2,6-di-F Ph | H | O |
| 21 | H | H | Me | 2,3-di-Cl Ph | H | O |
| 22 | H | H | Me | 3,4-di-Cl Ph | H | O |
| 23 | H | H | Me | 2,4-di-Cl Ph | H | O |
| 24 | H | H | Me | 2,6-di-Cl Ph | H | O |
| 25 | H | H | Me | 2,3-di-Me Ph | H | O |
| 26 | H | H | Me | 2,4-di-Me Ph | H | O |
| 27 | H | H | Me | 3,4-di-Me Ph | H | O |
| 28 | H | H | Me | 2,6-di-Me Ph | H | O |
| 29 | H | H | Me | 2,3-di-MeO Ph | H | O |
| 30 | H | H | Me | 2,4-di-MeO Ph | H | O |
| 31 | H | H | Me | 3,4-di-MeO Ph | H | O |
| 32 | H | H | Et | Ph | H | O |
| 33 | H | H | Et | 2-Cl Ph | H | O |
| 34 | H | H | Et | 2-Me Ph | H | O |
| 35 | H | H | Et | 3-F—Ph | H | O |
| 36 | H | H | Et | 3-Cl Ph | H | O |
| 37 | H | H | Et | 3-CF₃ Ph | H | O |
| 38 | H | H | Et | 3-Me Ph | H | O |
| 39 | H | H | Et | 3-MeO Ph | H | O |
| 40 | H | H | Et | 4-F—Ph | H | O |
| 41 | H | H | Et | 4-Cl Ph | H | O |
| 42 | H | H | Et | 4-CF₃ Ph | H | O |
| 43 | H | H | Et | 4-Me Ph | H | O |
| 44 | H | H | Et | 4-Et Ph | H | O |
| 45 | H | H | Et | 4-MeO Ph | H | O |
| 46 | H | H | Et | 4-EtO Ph | H | O |
| 47 | H | H | Et | 2,3-di-F Ph | H | O |
| 48 | H | H | Et | 2,4-di-F Ph | H | O |
| 49 | H | H | Et | 3,4-di-F Ph | H | O |
| 50 | H | H | Et | 2,6-di-F Ph | H | O |
| 51 | H | H | Et | 2,3-di-Cl Ph | H | O |
| 52 | H | H | Et | 2,4-di-Cl Ph | H | O |
| 53 | H | H | Et | 3,4-di-Cl Ph | H | O |
| 54 | H | H | Et | 2,6-di-Cl Ph | H | O |
| 55 | H | H | Et | 2,3-di-Me Ph | H | O |
| 56 | H | H | Et | 2,4-di-Me Ph | H | O |
| 57 | H | H | Et | 3,4-di-Me Ph | H | O |
| 58 | H | H | Et | 2,6-di-Me Ph | H | O |
| 59 | H | H | Et | 2,3-di-MeO Ph | H | O |
| 60 | H | H | Et | 2,4-di-MeO Ph | H | O |
| 61 | H | H | Et | 3,4-di-MeO Ph | H | O |
| 62 | H | H | iPr | Ph | H | O |
| 63 | H | H | iPr | 2-F Ph | H | O |
| 64 | H | H | iPr | 2-Cl Ph | H | O |
| 65 | H | H | iPr | 2-Me Ph | H | O |
| 66 | H | H | iPr | 2-MeO Ph | H | O |
| 67 | H | H | iPr | 3-F—Ph | H | O |
| 68 | H | H | iPr | 3-Cl Ph | H | O |
| 69 | H | H | iPr | 3-CF₃ Ph | H | O |
| 70 | H | H | iPr | 3-Me Ph | H | O |
| 71 | H | H | iPr | 3-MeO Ph | H | O |
| 72 | H | H | iPr | 4-F—Ph | H | O |
| 73 | H | H | iPr | 4-Cl Ph | H | O |
| 74 | H | H | iPr | 4-CF₃ Ph | H | O |
| 75 | H | H | iPr | 4-Me Ph | H | O |
| 76 | H | H | iPr | 4-Et Ph | H | O |
| 77 | H | H | iPr | 4-MeO Ph | H | O |
| 78 | H | H | iPr | 4-EtO Ph | H | O |
| 79 | H | H | iPr | 2,3-di-F Ph | H | O |
| 80 | H | H | iPr | 2,4-di-F Ph | H | O |
| 81 | H | H | iPr | 3,4-di-F Ph | H | O |
| 82 | H | H | iPr | 2,3-di-F Ph | H | O |
| 83 | H | H | iPr | 2,3-di-Cl Ph | H | O |
| 84 | H | H | iPr | 2,4-di-Cl Ph | H | O |
| 85 | H | H | iPr | 2,6-di-Cl Ph | H | O |
| 86 | H | H | iPr | 3,4-di-Cl Ph | H | O |
| 87 | H | H | iPr | 2,3-di-Me Ph | H | O |
| 88 | H | H | iPr | 2,4-di-Me Ph | H | O |
| 89 | H | H | iPr | 2,3-di-Me Ph | H | O |
| 90 | H | H | iPr | 2,3-di-Me Ph | H | O |
| 91 | H | H | iPr | 2,3-di-MeO Ph | H | O |
| 92 | H | H | iPr | 2,4-di-MeO Ph | H | O |
| 93 | H | H | iPr | 3,4-di-MeO Ph | H | O |
| 94 | Me | H | Et | 4-MeO Ph | H | O |
| 95 | Me | H | Et | 4-MeO Ph | H | S |
| 96 | H | H | Et | 4-MeO Ph | H | S |
| 97 | H | H | Me | 4-Et Ph | H | S |
| 98 | H | F | Et | 4-MeO Ph | H | O |
| 99 | H | H | Et | 4-MeO Ph | F | O |
| 100 | H | H | Et | 4-F—Ph | H | S |
| 101 | H | H | Et | 4-Cl Ph | H | S |
| 102 | H | H | Et | 4-CF₃ Ph | H | S |
| 103 | H | H | Et | 4-Me Ph | H | S |
| 104 | H | H | Et | 4-MeO Ph | H | S |
| 105 | H | H | Et | 4-EtO Ph | H | S |

The particular process used in the preparation of the compounds of the present disclosure depends upon the specific compound desired. Such factors as the selection of the specific moiety, and the specific substituents possible at various locations on the molecule, which all play a role in the path to be followed

In general, the compounds of this disclosure may be prepared by standard techniques known in the art and by known processes analogous thereto. For example, the compounds may be prepared according to methods described in U.S. Pat. No. 6,828,335, which is incorporated by reference in its entirety. The present disclosure also encompasses indane acetic acid compounds and derivatives described in U.S. Pat. Nos. 7,112,597, 8,541,618, and 8,552,203, each of which is incorporated by references in its entirety. The present disclosure also encompasses indane acetic acid derivatives and their use described in US Application Publication Number 2014/0086910, which is incorporated by reference in its entirety.

A salt of a compound described in the present disclosure may be prepared in situ during the final isolation and purification of a compound or by separately reacting the purified compound in its free base form with a suitable organic or inorganic acid and isolating the salt thus formed. Likewise, when the compound described in the present disclosure contains a carboxylic acid moiety, (e.g., R=H), a salt of said compound may be prepared by separately reacting it with a suitable inorganic or organic base and isolating the salt thus formed. The term “pharmaceutically acceptable salt” refers to a relatively non-toxic, inorganic or organic acid addition salt of a compound of the present disclosure (See, e.g., Berge et al., J. Pharm. Sci. 66:1-19, 1977, incorporated herein with regard to formation of salts).

Representative salts of the compounds described in the present disclosure include the conventional non-toxic salts and the quaternary ammonium salts, which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, undecanoate, and the like.

Base salts include, for example, alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups in the conjugate base may be quaternized with alkyl halides, e.g., C₁₋₉ alkyl halides such as methyl, ethyl, propyl, and butyl chlorides, bromides and iodides; dialkyl sulfates like dimethyl, diethyl, and dibutyl sulfate; and diamyl sulfates, C₁₀₋₄₀ alkyl halides such as decyl, lauryl, myristyl and stearyl chlorides, bromides and iodides; or aralkyl halides like benzyl and phenethyl bromides. In some embodiments, the salts are alkali salt such as sodium or potassium salt or an adduct with an acceptable nitrogen base such as meglumine (N-Methyl-d-glucamine) salt.

The esters of the compounds described in the present disclosure are non-toxic, pharmaceutically acceptable esters, for example, alkyl esters such as methyl, ethyl, propyl, isopropyl, butyl, isobutyl, or pentyl esters. Additional esters such as, for example, methyl ester or phenyl-C₁-C₅ alkyl may be used. The compounds described in the present disclosure may be esterified by a variety of conventional procedures including reacting the appropriate anhydride, carboxylic acid, or acid chloride with the alcohol group of the compounds described in the present disclosure. The appropriate anhydride may be reacted with the alcohol in the presence of a base to facilitate acylation such as 1,8-bis[dimethylamino]naphthalene or N,N-dimethylaminopyridine. An appropriate carboxylic acid may be reacted with the alcohol in the presence of a dehydrating agent such as dicyclohexylcarbodiimide, 1-[3-dimethylaminopropyl]-3-ethylcarbodiimide, or other water soluble dehydrating agents which are used to drive the reaction by the removal of water, and optionally, an acylation catalyst. Esterification may also be effected using the appropriate carboxylic acid in the presence of trifluoroacetic anhydride and optionally, pyridine, or in the presence of N, N-carbonyldiimidazole with pyridine. Reaction of an acid chloride with the alcohol may be carried out with an acylation catalyst such as 4-DMAP or pyridine.

One skilled in the art would readily know how to successfully carry out these, as well as other methods of esterification of alcohols.

Additionally, sensitive or reactive groups on the compound described in the present disclosure may need to be protected and deprotected during any of the above methods for forming esters.

The compounds described in the present disclosure may contain one or more asymmetric centers, depending upon the location and nature of the various substituents desired. Asymmetric carbon atoms may be present in the (R) or (S) configuration. Preferred isomers are those with the absolute configuration, which produces the compound of described in the present disclosure with the more desirable biological activity. In certain instances, asymmetry may also be present due to restricted rotation about a given bond, for example, the central bond adjoining two aromatic rings of the specified compounds.

Substituents on a ring may also be present in either cis or trans form, and a substituent on a double bond may be present in either Z or E form.

It is intended that all isomers (including enantiomers and diastereomers), either by nature of asymmetric centers or by restricted rotation as described above, as separated, pure or partially purified isomers or racemic mixtures thereof, be included within the scope of the instant disclosure. The purification of said isomers and the separation of said isomeric mixtures may be accomplished by standard techniques known in the art.

As described herein, compounds of the disclosure may optionally be substituted with one or more substituents, such as are illustrated generally above, or as exemplified by particular classes, subclasses, and species of the disclosure. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds.

### Pharmaceutical Compositions

According to another aspect of the present disclosure, pharmaceutical compositions of compounds described herein are provided. In some embodiments, the pharmaceutical compositions further include a pharmaceutically acceptable carrier.

In some embodiments, the pharmaceutical compositions described herein may further include one or more additional therapeutic agents.

In one embodiment, the additional therapeutic agents are direct acting SARS-CoV-2 anti-viral agents, such as, for example, remidesivir, nafamostat, or ribaviran.

In one embodiment, the additional therapeutic agent is selected from one or more of chloroquine and hydroxychloroquine.

Based on well-known assays used to determine the efficacy for treatment of conditions identified above in mammals, and by comparison of these results with the results of known medicaments that are used to treat these conditions, the effective dosage of the compounds of this disclosure may readily be determined for treatment of each desired indication. The amount of the active ingredient (e.g., compounds) to be administered in the treatment of one of these conditions may vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 10 mg/kg, and preferably from about 0.001 mg/kg to about 10 mg/kg body weight per day. A unit dosage may contain from about 0.05 mg to about 500 mg of active ingredient, and may be administered one or more times per day. The daily dosage for administration by injection, including intravenous, intramuscular, subcutaneous, and parenteral injections, and use of infusion techniques may be from about 0.0001 mg/kg to about 10 mg/kg. The daily rectal dosage regimen may be from 0.0001 mg/kg to 10 mg/kg of total body weight. The transdermal concentration may be that required to maintain a daily dose of from 0.0001 mg/kg to 10 mg/kg.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present disclosure may be ascertained by those skilled in the art using conventional treatment tests.

The compounds of this disclosure may be utilized to achieve the desired pharmacological effect by administration to a patient in need thereof in an appropriately formulated pharmaceutical composition. A patient, for the purpose of this disclosure, is a mammal, including a human, in need of treatment for a particular condition or disease. Therefore, the present disclosure includes pharmaceutical compositions which include a pharmaceutically acceptable carrier and a therapeutically effective amount of a compound. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a compound is that amount which produces a result or exerts an influence on the particular condition being treated. The compounds described herein may be administered with a pharmaceutically-acceptable carrier using any effective conventional dosage unit forms, including, for example, immediate and timed release preparations, orally, parenterally, topically, or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which may be of the ordinary hard- or soft-shelled gelatin type containing, for example, surfactants, lubricants, and inert fillers such as lactose, sucrose, calcium phosphate, and corn starch.

In another embodiment, the compounds of this disclosure may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this disclosure may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soybean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this disclosure may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which may be used in the parenteral formulations of this disclosure are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quaternary ammonium salts, as well as mixtures.

The parenteral compositions of this disclosure may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant may be a single component having the above HLB or may be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the disclosure may also be administered in the form of suppositories for rectal administration of the drug. These compositions may be prepared by mixing the drug (e.g., compound) with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present disclosure employs transdermal delivery devices (“patches”). Such transdermal patches may be used to provide continuous or discontinuous infusion of the compounds of the present disclosure in controlled amounts. The construction and use of transdermal patches for the delivery of pharmaceutical agents is well known in the art (See, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the disclosure may also contain other conventional pharmaceutically acceptable compounding ingredients, generally referred to as carriers or diluents, as necessary or desired. Any of the compositions of this disclosure may be preserved by the addition of an antioxidant such as ascorbic acid or by other suitable preservatives. Conventional procedures for preparing such compositions in appropriate dosage forms may be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, or trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (includes but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, octoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds described herein may be administered as the sole pharmaceutical agent or in combination with one or more other pharmaceutical agents where the combination causes no unacceptable adverse effects. One potential combination includes one or more direct acting antivirals. Another potential combination includes one or more therapeutics that alleviate inflammation. Potential combinations include one or more of remdesivir, niclosamide, favipiravir (favilavir, Avigan), nafamostat, camostat, galidesivir, Jakafi (ruxolitinib), losartan (angiotensin II receptor antagonist), and tocilizumab.

The compounds described herein may also be utilized, in free base form or in compositions, in research and diagnostics, or as analytical reference standards, and the like. Therefore, the present disclosure includes compositions which include an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

The compounds may be administered to subjects by any suitable route, including orally (inclusive of administration via the oral cavity), parenterally, by inhalation spray, topically, transdermally, rectally, nasally, sublingually, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, parenterally, transdermally or by inhalation spray.

It should also be understood that a specific dosage and treatment regimen for any particular patient will depend upon a variety of factors, including the activity of the specific compound employed, the age, body weight, general health, gender, diet, time of administration, rate of excretion, drug combination, and the judgment of the treating physician and the severity of the particular disease being treated. The amount of a compound of the present disclosure in the composition will also depend upon the particular compound in the composition.

The following examples are presented to illustrate the disclosure described herein, but should not be construed as limiting the scope of the disclosure in any way.

Capsule Formulation

A capsule formula is prepared from:

| | |
|---|---|
| Compound of this disclosure | 10 mg |
| Starch | 109 mg |
| Magnesium stearate | 1 mg |

The components are blended, passed through an appropriate mesh sieve, and filled into hard gelatin capsules.

Tablet Formulation

A tablet is prepared from:

| | |
|---|---|
| Compound of this disclosure | 25 mg |
| Cellulose, microcrystalline | 200 mg |
| Colloidal silicon dioxide | 10 mg |
| Stearic acid | 5.0 mg |

The ingredients are mixed and compressed to form tablets. Appropriate aqueous and non-aqueous coatings may be applied to increase palatability, improve elegance and stability or delay absorption.

Sterile IV Solution

A mg/mL solution of the desired compound of this disclosure is made using sterile, injectable water, and the pH is adjusted if necessary. The solution is diluted for administration with sterile 5% dextrose and is administered as an IV infusion.

Intramuscular Suspension

The following intramuscular suspension is prepared:

| | |
|---|---|
| Compound of this disclosure | 50 mg/mL |
| Sodium carboxymethylcellulose | 5 mg/mL |
| TWEEN 80 | 4 mg/mL |
| Sodium chloride | 9 mg/mL |
| Benzyl alcohol | 9 mg/mL |

The suspension is administered intramuscularly.

Hard Shell Capsules

A large number of unit capsules are prepared by filling standard two-piece hard galantine capsules each with powdered active ingredient, 150 mg of lactose, 50 mg of cellulose, and 6 mg of magnesium stearate.

Soft Gelatin Capsules

A mixture of active ingredients in a digestible oil, such as soybean oil, cottonseed oil, or olive oil, is prepared and injected by means of a positive displacement pump into molten gelatin to form soft gelatin capsules containing the active ingredient. The capsules are washed and dried. The active ingredient may be dissolved in a mixture of polyethylene glycol, glycerin and sorbitol to prepare a water miscible medicine mix.

Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin, and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Methods of Use

One embodiment of the present disclosure includes a method for treating a subject having a beta coronavirus infection comprising administering a therapeutically effective amount of a Peroxisome Proliferator-Activated Receptor (PPAR) agonist, which penetrates the blood brain barrier (BBB).

One aspect of the present disclosure includes wherein the betacoronovirus is selected from one or more of SARS-CoV-2, SARS-CoV-1, MERS-CoV, NCoV-OC43, HCoV-HKU1, and a novel beta coronavirus.

One aspect of the present disclosure includes wherein the beta coronavirus infection causes or exacerbates one or more of Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), and elevated blood glucose levels.

One aspect of the present disclosure includes wherein the therapeutically effective amount provides pharmacologically useful concentrations in the brain.

One aspect of the present disclosure includes wherein the PPAR agonist is a PPAR-delta agonist, a PPAR-gamma agonist, or a dual PPAR delta and gamma agonist.

According to one aspect of the present disclosure, includes methods of treating a beta coronavirus infection including COVID-19 disease ARDS and cognitive impairment in COVID-19 disease. The methods include administering to a subject in need of such treatment an effective amount of a compound of the present disclosure. In some embodiments, the compound is administered intravenously, orally, buccally, transdermally, rectally, nasally, optically, intrathecally, or intra-cranially.

In another embodiment, the compounds of the present disclosure may be administered in combination with one or more additional therapeutic agent. One potential combination includes one or more direct acting antivirals. Another potential combination includes one or more therapeutics that alleviate inflammation. Potential combinations include one or more of remdesivir, niclosamide, favipiravir (favilavir, Avigan), nafamostat, camostat, galidesivir, Jakafi (ruxolitinib), losartan (angiotensin II receptor antagonist), and tocilizumab. Exemplary additional therapeutic agents include chloroquine, hydroxychloroquine, remdesivir, or nafamostat mesylate. The compounds described herein may be administered in combination with one or more further medicaments of use for the treatment or prevention of the listed conditions and disease.

Depending on the individual medicaments utilized in a combination therapy for simultaneous administration, they may be formulated in combination (where a stable formulation may be prepared and where desired dosage regimes are compatible) or the medicaments may be formulated separately (for concomitant or separate administration through the same or alternative routes).

In some embodiments, the subject of the present disclosure possesses one or more risk factors for developing COVID-19 disease selected from a family history of the disease; obesity, insulin resistance and Type 2 Diabetes Mellitus, high cholesterol, high triglycerides, and metabolic syndrome.

### EXAMPLES

Embodiments of the present disclosure will now be described by way of example only with respect to the following non-limiting examples.

In general, the compounds of this disclosure may be prepared by standard techniques known in the art and by known processes analogous thereto. For example, the compounds may be prepared according to methods described in U.S. Pat. No. 6,828,335, which is incorporated by reference in its entirety.

### Example 1

### Ethyl [(1S)-5-hydroxy-2,3-dihydro-1H-inden-1-yl]acetate

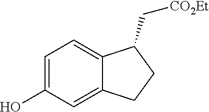 [SMILES: CCOC(=O)C[C@@H]1CCc2cc(O)ccc21]

Prepared in six steps from 5-methoxy indanone as described in U.S. Pat. No. 6,828,335.

### Example 2

### 2-[5-ethyl-2-(4-methoxyphenyl)-1,3-oxazol-4-yl]ethanol

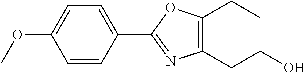 [SMILES: CCc1oc(-c2ccc(OC)cc2)nc1CCO]

Prepared from L-aspartic acid n-methyl ester hydrochloride, 4-methoxy benzoyl chloride and proprionic anhydride as generally described in U.S. Pat. No. 6,828,335.

### Example 3

### 2-[2-(4-methoxyphenyl)-5-methyl-1,3-oxazol-4-yl]ethanol

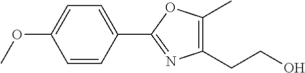 [SMILES: COc1ccc(-c2nc(CCO)c(C)o2)cc1]

Prepared from L-aspartic acid β-methyl ester hydrochloride, 4-methoxy benzoyl chloride and acetic anhydride as generally described in U.S. Pat. No. 6,828,335.

### Example 4

### 2-[5-Ethyl-2-(4-methylphenyl)-1,3-oxazol-4-yl]ethanol

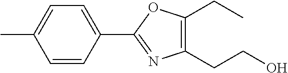 [SMILES: CCc1oc(-c2ccc(C)cc2)nc1CCO]

Prepared from L-aspartic acid β-methyl ester hydrochloride, p-toluoyl chloride and proprionic anhydride as generally described in U.S. Pat. No. 6,828,335.

### Example 5

### 2-[5-Methyl-2-(4-methylphenyl)-1,3-oxazol-4-yl]ethanol

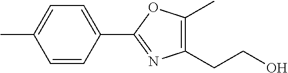 [SMILES: Cc1ccc(-c2nc(CCO)c(C)o2)cc1]

Prepared as from L-aspartic acid β-methyl ester hydrochloride, p-toluoyl chloride and acetic anhydride as described in U.S. Pat. No. 6,828,335.

### Example 6

### 2-[5-Ethyl-2-(4-ethylphenyl)-1,3-oxazol-4-yl]ethanol

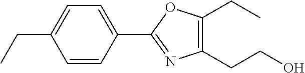 [SMILES: CCc1ccc(-c2nc(CCO)c(CC)o2)cc1]

Prepared from L-aspartic acid β-methyl ester hydrochloride, 4-ethyl benzoyl chloride and proprionic anhydride as generally described in U.S. Pat. No. 6,828,335.

### Example 7

### 2-[2-(4-Ethylphenyl)-5-methyl-1,3-oxazol-4-yl]ethanol

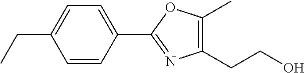 [SMILES: CCc1ccc(-c2nc(CCO)c(C)o2)cc1]

Prepared from L-aspartic acid β-methyl ester hydrochloride, 4-ethyl benzoyl chloride and acetic anhydride as generally described in U.S. Pat. No. 6,828,335.

### Example 8

### 2-(5-ethyl-2-(4-methoxyphenyl)oxazol-4-yl)ethyl benzenesulfonate

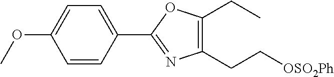

The intermediate from Example 2 (400.8 g), 15.0 g trimethylamine hydrochloride and 3.2 L dichloromethane was added to a 22 L reactor. The reaction mixture was stirred and cooled to 3.8° C. 680 mL of 41 rimethylamine was then added to the reactor. Benzenesulfonyl chloride (400 g) is slowly added to the reactor while maintaining the temperature below 12° C. The reaction was cooled to between 5° C. and 10° C. for three hours and then heated to 20° C. The contents of the reactor were stirred overnight at 24° C. Additional 3.2 L of dichloromethane was added to the reactor. The mixture was cooled to 5.0° C. and 205 mL 3-dimethylamino-1-propylamine was added. The mixture is stirred at 4.8° C. for 16 minutes. An aqueous citric acid solution (3 L of 1M) was slowly added to the reactor so as to maintain the temperature below 16° C. The resulting mixture was heated to 20° C. and stirred for 10 minutes. The phases were separated, and the organics were washed with 3 L of 1M citric acid solution, 3 L saturated sodium bicarbonate solution, 3 L brine solution, dried with magnesium sulfate, filtered and concentrated. The residue was treated with n-heptane and concentrated to give 542 g of crude 2-(5-ethyl-2-(4-methoxyphenyl)oxazol-4-yl)ethyl benzenesulfonate.

### Example 9

### (S)-Ethyl 2-(5-(2-(5-ethyl-2-(4-methoxyphenyl)oxazol-4-yl)ethoxy)-2,3-dihydro-1H-inden-1-yl)acetate

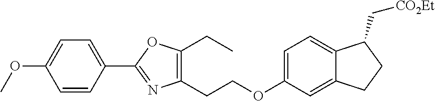 [SMILES: CCOC(=O)C[C@@H]1CCc2cc(OCCc3nc(-c4ccc(OC)cc4)oc3CC)ccc21]

A 22 L reactor was charged with 302.3 g of ethyl [(1S)-5-hydroxy-2,3-dihydro-1H-inden-1-yl]acetate (Example 1), 539.3 g crude 2-(5-ethyl-2-(4-methoxyphenyl)oxazol-4-yl)ethyl benzenesulfonate (Example 8) and 3.4 L acetonitrile. The mixture was stirred until all of the solids dissolved; then, 670.6 g cesium carbonate was added. The mixture is heated to 70° C. and held 16 hours. An additional charge of 60.2 g of compound from Example 1 was added to the reactor. The mixture was heated to 70° C. for one hour and additional cesium carbonate (316.9 g) was added and heating was continued for 2.5 hours at 70° C. The reaction mixture was cooled to 24° C. and 4 L n-heptane, 2.4 L USP water, 2.4 L brine solution and 4 L ethyl acetate was charged to the reactor. The biphasic mixture was stirred for 5 minutes, then allowed to separate. The organic layer was washed with 2×2.4 L 5% sodium hydroxide solution and 2.4 L USP water, and 2.4 L brine. The solvent is removed via rotary evaporation until solids precipitate. Addition of 7.7 L n-heptane and stirring produced a slurry, which was filtered, and the filter cake was rinsed with the filtrate and then with 2.4 L n-heptane. The product air dried and then dried in a vacuum oven at 40° C. to give (S)-ethyl 2-(5-(2-(5-ethyl-2-(4-methoxyphenyl)oxazol-4-yl)ethoxy)-2,3-dihydro-1H-inden-1-yl)acetate as an off white solid.

### Example 10

### (S)-2-(5-(2-(5-ethyl-2-(4-methoxyphenyl)oxazol-4-yl)ethoxy)-2,3-dihydro-1H-inden-1-yl)acetic acid

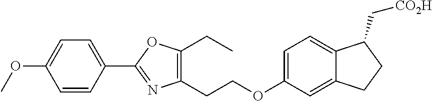 [SMILES: CCc1oc(-c2ccc(OC)cc2)nc1CCOc1ccc2c(c1)CC[C@H]2CC(=O)O]

A 22 L flask was charged with 478.9 g of (S)-ethyl 2-(5-(2-(5-ethyl-2-(4-methoxyphenyl)oxazol-4-yl)ethoxy)-2,3-dihydro-1H-inden-1-yl)acetate (Example 9) and 1.2 L ethanol and cooled to 20° C. To the 22 L flask was charged 1.6 L of 1N sodium hydroxide solution. The reaction mixture was heated to 65° C. for 30, then cooled to 25° C., and concentrated to an oil. A new reaction flask was charged with 4.8 L USP water and 1.9 L 1N hydrochloric acid solution, vigorously stirred and cooled to 23° C. The product oil was added to the solution via an addition funnel. The resulting suspension is stirred at approximately 23° C., and the pH is checked: 1.6 (target 2). The solids were filtered and then washed with the mother liquor. The solids were washed with 3 L USP water and then with 1.9 L 1:1 ethanol SDA-2B:water. The filter cake was air dried for 4 hours and is then transferred to a vacuum oven. The solid was dried under vacuum at 45° C. until a constant mass was achieved, producing (S)-2-(5-(2-(5-ethyl-2-(4-methoxyphenyl)oxazol-4-yl)ethoxy)-2,3-dihydro-1H-inden-1-yl)acetic acid as an off white solid.

### Example 11

### Sodium (S)-2-(5-(2-(5-ethyl-2-(4-methoxyphenyl)oxazol-4-yl)ethoxy)-2,3-dihydro-1H-inden-1-yl)acetate

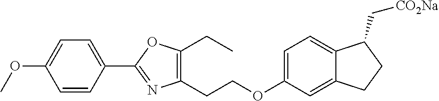

A 22 L reactor was charged with 3.8 L ethanol. Agitation was started, and the reactor was charged successively with 288.2 g sodium ethoxide solution (20.1% in ethanol) and with 378.4 g of (S)-2-(5-(2-(5-ethyl-2-(4-methoxyphenyl)oxazol-4-yl)ethoxy)-2,3-dihydro-1H-inden-1-yl)acetic acid (Example 10). The reaction mixture was heated to 40° C. for −20 minutes (until all solids are dissolved), and pH was checked (target pH 9-10). The solution was filtered through a 10 micron filter membrane, returned to the reactor and heated to 40° C. The reactor was then charged with 3.4 L of filtered methyl t-butyl ether at such a rate that the temperature of the product solution is maintained at 40° C. throughout. The mixture is then seeded with 0.5 g Example 10 compound, and held at 42° C. for 40 minutes. An additional 3.4 L of filtered methyl t-butyl ether was added. The suspension was heated to 55° C. for 65 minutes. The suspension was cooled to 20-25° C. overnight then to 14° C. the next morning. The product was filtered under a nitrogen blanket, washed with 1.3 L filtered methyl t-butyl ether and dried to constant mass in a vacuum oven at 40° C. The bulk product was milled using a Comil with a 10 mesh sieve. The product is dried in a humidified environment at 40° C. NMR analysis showed ≤0.5% of ethanol by weight. Final product sodium (S)-2-(5-(2-(5-ethyl-2-(4-methoxyphenyl)oxazol-4-yl)ethoxy)-2,3-dihydro-1H-inden-1-yl)acetate was further dried at 45° C. under vacuum to obtain 306 g as a fine white solid. Example 11 may be referred to as T3D-959.

### Example 12

### (S)-2-(5-(2-(2-(4-methoxyphenyl)-5-methyloxazol-4-yl)ethoxy)-2,3-dihydro-1H-inden-1-yl)acetic Acid

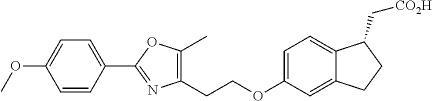 [SMILES: COc1ccc(-c2nc(CCOc3ccc4c(c3)CC[C@H]4CC(=O)O)c(C)o2)cc1]

(S)-Ethyl 2-(5-hydroxy-2,3-dihydro-1H-inden-1-yl)acetate from Example 1 and 2-(2-(4-methoxyphenyl)-5-methyloxazol-4-yl)ethanol from Example 3 were combined and reacted as in Examples 8, 9 and 10 to give (S)-2-(5-(2-(2-(4-methoxyphenyl)-5-methyloxazol-4-yl)ethoxy)-2,3-dihydro-1H-inden-1-yl)acetic acid as an off white solid.

### Example 13

### (S)-2-(5-(2-(5-ethyl-2-p-tolyloxazol-4-yl)ethoxy)-2,3-dihydro-1H-inden-1-yl)acetic Acid

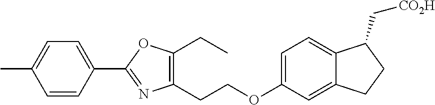 [SMILES: CCc1oc(-c2ccc(C)cc2)nc1CCOc1ccc2c(c1)CC[C@H]2CC(=O)O]

(S)-Ethyl 2-(5-hydroxy-2,3-dihydro-1H-inden-1-yl)acetate from Example 1 and 2-(5-ethyl-2-p-tolyloxazol-4-yl)ethanol from Example 4 were combined and reacted as in Examples 8, 9 and 10 to give (S)-2-(5-(2-(5-ethyl-2-p-tolyloxazol-4-yl)ethoxy)-2,3-dihydro-1H-inden-1-yl)acetic acid as an off white solid.

### Example 14

### (S)-2-(5-(2-(5-methyl-2-p-tolyloxazol-4-yl)ethoxy)-2,3-dihydro-1H-inden-1-yl)acetic Acid

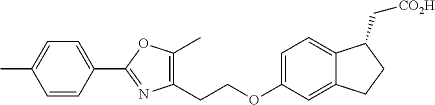 [SMILES: Cc1ccc(-c2nc(CCOc3ccc4c(c3)CC[C@H]4CC(=O)O)c(C)o2)cc1]

(S)-Ethyl 2-(5-hydroxy-2,3-dihydro-1H-inden-1-yl)acetate from Example 1 and 2-(5-methyl-2-p-tolyloxazol-4-yl)ethanol from Example 5 were combined and reacted as described in Examples 8, 9 and 10 to give (S)-2-(5-(2-(5-methyl-2-p-tolyloxazol-4-yl)ethoxy)-2,3-dihydro-1H-inden-1-yl)acetic acid as an off white solid.

### Example 15

### (S)-2-(5-(2-(5-ethyl-2-(4-ethylphenyl)oxazol-4-yl)ethoxy)-2,3-dihydro-1H-inden-1-yl)acetic Acid

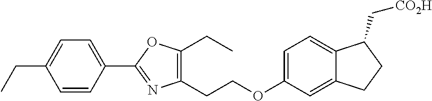 [SMILES: CCc1ccc(-c2nc(CCOc3ccc4c(c3)CC[C@H]4CC(=O)O)c(CC)o2)cc1]

(S)-Ethyl 2-(5-hydroxy-2,3-dihydro-1H-inden-1-yl)acetate from Example 1 and 2-(5-ethyl-2-(4-ethylphenyl)oxazol-4-yl)ethanol from Example 6 were combined and reacted as described in Examples 8, 9 and 10 to give (S)-2-(5-(2-(5-ethyl-2-(4-ethylphenyl)oxazol-4-yl)ethoxy)-2,3-dihydro-1H-inden-1-yl)acetic acid as an off white solid.

### Example 16

### (S)-2-(5-(2-(5-ethyl-2-(4-ethylphenyl)oxazol-4-yl)ethoxy)-2,3-dihydro-1H-inden-1-yl)acetic Acid

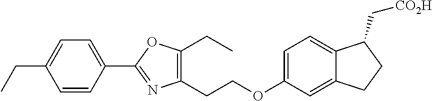 [SMILES: CCc1ccc(-c2nc(CCOc3ccc4c(c3)CC[C@H]4CC(=O)O)c(CC)o2)cc1]

(S)-Ethyl 2-(5-hydroxy-2,3-dihydro-1H-inden-1-yl)acetate from Example 1 and 2-(2-(4-ethylphenyl)-5-methyloxazol-4-yl)ethanol from Example 7 were combined and reacted as described in Examples 8, 9 and 10 to give (S)-2-(5-(2-(2-(4-ethylphenyl)-5-methyloxazol-4-yl)ethoxy)-2,3-dihydro-1H-inden-1-yl)acetic acid as an off white solid.

### Evaluation of Biological Activity of Compounds

Demonstration of the activity of the compounds of the present disclosure may be accomplished through in vitro, ex vivo and in vivo assays that are well known in the art or as herein described.

PPAR receptor agonist activity may be determined by conventional screening methods known to the skilled in the art. For example, methods described in U.S. Patent Application Publication No. 2007/0054907, 2008/0262047 and U.S. Pat. No. 7,314,879, which are incorporated by reference in their entireties.

### Animal Models of Beta Coronavirus Disease

The compounds described in the present disclosure may be tested in any animal model known to those skilled in the art. For each model, the test result may be compared with a control group that is not treated. The treated animals may demonstrate an improvement in the performance of a variety of tests. The improvement may be straightforward or nuanced.

### Clinical Trial (Prophetic)

The compounds described in the present disclosure may be tested clinically in randomized clinical trials in COVID-19 positive patients.

A prophetic COVID-19 Clinical Trial may include the following parameters. The trial would be randomized and placebo-controlled. The subjects would be newly hospitalized with confirmed COVID-19 diagnosis. The trial would have at least 2 arms, with 2 arms (1:1 ratio): placebo vs. 30 mg T3D-959. The proposed dose administration would be oral (2 capsules) q.d. for 28-days. The propose outcome measure options, either primary or secondary, include one or more of:
1. Survival
2. Length of Hospital Stay
3. Rate of mechanical ventilation
4. Time to sero-negativity
5. COVID Ordinal Outcomes Scale (various timepoints)
6. Oxygen-free days [Time Frame: about 28 days after randomization]
7. Ventilator-free days [Time Frame: about 28 days after randomization]
8. ICU-free days [Time Frame: about 28 days after randomization]
9. Hospital-free days [Time Frame: about 28 days after randomization]

Data would be analyzed for statistical significance, such as an appropriate ANOVA design evaluation.

### Test Results

### Example 17

### Human PPAR Activation by Compound of Example 11 in Transient Transfection Study

Table 2 summarizes the results of studies performed in transfected CV-1 cells with three different lots of Compound of Example 11. These results showed an average EC₅₀ for activation of the human gamma subtype of 297 nM with a 74% maximal response. In similar experiments rosiglitazone had a human gamma subtype EC₅₀ of 130 nM. The average EC₅₀ for activation of the human δ subtype was 19 nM with a 76% maximal response. In similar experiments GW501516 had a human δ subtype EC₅₀ of 1.3 nM. The average EC₅₀ for activation of human alpha subtype was 530 nM with a significantly reduced maximal response. These results demonstrate that (S)-2-(5-(2-(5-ethyl-2-(4-methoxyphenyl)oxazol-4-yl)ethoxy)-2,3-dihydro-1H-inden-1-yl)acetate is a potent, selective agonist of the PPARδ and PPARδ subtypes, with a 15-fold greater potency in activating the human PPARδ subtype over the human PPARγ subtype and about a 30-fold selectivity over the human PPAR+balpha subtype.

TABLE 2
Summary of Human PPAR Activation by Compound of
Example 11 in Transient Transfection Studies

| | Material | Use | PPARγ | PPARδ | PPARα |
|---|---|---|---|---|---|
| EC50 | Lot A | Non GMP | 220 | 15 | 488 |
| (nM) | Lot B | GLP Nonclinical | 270 | 27 | 750 |
| | | Studies | | | |
| | Lot C | cGMP material | 400 | 16 | 360 |

| | | | |
|---|---|---|---|
| Average EC₅₀ (nM) | 297 | 19 | 530 |
| Average % maximal response | 74 | 76 | 49 |
| Peak effect | 29.49 | 17.68 | 1.87 |

### Example 18: Effect of P-gp Inhibitor Verapamil on Caco-2 Permeability of Compound of Example 11

The human Caco-2 permeability of Compound of Example 11 was evaluated. High permeability of (S)-2-(5-(2-(5-ethyl-2-(4-methoxyphenyl)oxazol-4-yl)ethoxy)-2,3-dihydro-1H-inden-1-yl)acetate was observed in the absence of a P-glycoprotein (P-gp) inhibitor (Papparent=1144 nm/sec); no significant change in permeability was observed in the presence of the P-gp inhibitor (verapamil). These results indicate that (S)-2-(5-(2-(5-ethyl-2-(4-methoxyphenyl)oxazol-4-yl)ethoxy)-2,3-dihydro-1H-inden-1-yl)acetate is not a substrate for P-gp.

### Example 19

### Determination of Brain to Plasma Ratios for Compound of Example 11

Over 98% of drugs in clinical development for all diseases fail to adequately penetrate the blood brain barrier (BBB) to provide adequate brain exposure. For compounds of the present disclosure to be effective in treating cognitive impairment, it must have an ability to cross the BBB and penetrate the brain. To assess the ability of compounds of the present disclosure to cross the BBB, the pharmacokinetics and brain-to-plasma ratio of compound of Example 11, (Sodium (S)-2-(5-(2-(5-ethyl-2-(4-methoxyphenyl)oxazol-4-yl)ethoxy)-2,3-dihydro-1H-inden-1-yl)acetate was evaluated after oral dosing in male Sprague-Dawley rats. The test compound was dosed at 3 mg/kg from normal saline. Plasma and brain levels were determined by LC-MS/MS at pre-determined time points. Pharmacokinetic parameters were estimated by a non-compartmental model using WinNonlin v5.3 software. After oral dosing of test compound at 3 mg/kg, plasma Cₘₐₓ values of 1547±248 ng/mL were reached at 5 hours post dose. The average plasma half-life was 3.33 hours. The plasma exposure as measured by AUCₗₐₛₜ was 9569±1190 hr*ng/mL. Brain/Plasma ratios were found to be 0.361±0.142, 0.220±0.033, 0.171±0.011, 0.328±0.154, and 0.350±0.077 at 1, 3, 5, 7, and 12 hours, respectively. Results from the 15 rat experiment are shown in Table 3 below.

TABLE 3
Rat Brain Penetrance Pharmacokinetics
of Compound of Example 11

| Time | | Plasma | Brain Tissue | B/P | Average | |
|---|---|---|---|---|---|---|
| point (hr) | Rat# | Conc. (ng/mL) | Cone. (ng/g) | Ratio | (ng/g) | SD |
| 1.0 | 840 | 453 | 161 | 0.36 | 0.361 | 0.142 |
| | 841 | 199 | 101 | 0.51 | | |
| | 842 | 1020 | 226 | 0.22 | | |
| 3.0 | 843 | 910 | 234 | 0.26 | 0.220 | 0.033 |
| | 844 | 1270 | 269 | 0.21 | | |
| | 845 | 1430 | 274 | 0.19 | | |
| 5.0 | 846 | 1260 | 208 | 0.17 | 0.171 | 0.011 |
| | 847 | 1340 | 247 | 0.18 | | |
| | 848 | 2040 | 333 | 0.16 | | |
| 7.0 | 849 | 1240 | 203 | 0.16 | 0.328 | 0.154 |
| | 850 | 368 | 130 | 0.35 | | |
| | 851 | 462 | 216 | 0.47 | | |
| 12.0 | 852 | 345 | 104 | 0.30 | 0.350 | 0.077 |
| | 853 | 259 | 114 | 0.44 | | |
| | 854 | 377 | 117 | 0.31 | | |

### Example 20

Fasting Plasma Glucose Levels for Compound 11 from a Clinical Study

In a 2-week clinical study compound of Example 11 in 34 subjects, Fasting Plasma Glucose (FPG) levels were lowered as a function of dose. FPG measured in millimoles/Liter (MMOL/L), averaged over all subjects, was lowered from day 1 to day 14. For each individual dose group day 14 FPG levels were lower than day 1 levels. In addition, the decrease was inversely proportional to dose, such that the largest decrease in FPG was observed for the highest dose. For the two highest doses, the FPG remained lower on follow-up testing on day 21, one week after end of treatment.

TABLE 4
Clinical Fasting Plasma Glucose Levels for Compound 11

| | FPG on | FPG on | FPG on | Change from | Change from |
|---|---|---|---|---|---|
| Dose | Day 1 | Day 14 | Day 21 | D 1 to D 14 | D 1 to D 21 |
| Group | (MMOL/L) | (MMOL/L) | (MMOL/L) | (MMOL/L) | (MMOL/L) |
| All subjects | 99.1 | 94.8 | 98.1 | −4.3 | −1 |
| 3 mg | 94.6 | 92.4 | 97.1 | −2.2 | 2.5 |
| 10 mg | 101.4 | 98 | 101.9 | −3.4 | 0.5 |
| 30 mg | 99.2 | 94.6 | 99.1 | −4.6 | −0.1 |
| 90 mg | 101.1 | 94.1 | 96.4 | −7.0 | −4.7 |

### Example 21

### Clinical DSST Data in Cognitively Impaired Subjects

DSST is a commonly used tests in clinical neuropsychology, and has been used to measure a range of cognitive functions including intact motor speed, attention, executive, and visuo-perceptual functions. Reference is made to Jaeger J. Digit Symbol Substitution Test: The Case for Sensitivity Over Specificity hi Neuropsychological Testing. J Clin Psychopharmacol. 2018; 38(5):513-519. doi:10.1097/JCP.0000000000000941, which is incorporated by reference with regard to the example. DSST scores were assessed in a randomized, parallel, 4-dose Phase 2a study in subjects with mild-to-moderate dementia (MMSE for mild 20-26, for moderate 14-19) before and after treatment with compound of Example 11. A total of 34 subjects were randomized to one of 4 doses of drug, 3 mg (n=8), 10 mg (n=9), 30 mg (n=9) or 90 mg (n=8) administered orally once-a-day for 14 days then followed-up at day 21 (7-days post-dosing discontinuation.

DSST scores are based on the number of correct substitutions in a fixed timeframe, and a higher score in the DSST denotes improvement. Data was collected at four time points for all subjects in each of the four dose groups: Prescreen (PS, one week before BL), baseline (BL), end-of-treatment (EOT) and follow-up (FU, one week after EOT). The DSST results averaged across all subjects at PS (14.1, SD=11.8) and BL (13.9, SD=12.5) were quite similar as would be expected. The larger standard deviations, and the wide range of scores (zero to 48) reflects the broad range of cognitive function in the 34 subjects in this trial. DSST averages per dose group at PS were: 3 mg, 13.88 (SD=10.83); 10 mg, 14.33 (SD=13.52); 30 mg, 18.78 (SD=14.52); and 90 mg, 8.88 (SD=6.56). The data is best described as averaged differences in individual results as it reduces variability. For all 34 subjects, the change in DSST score was −0.21 (SD=4.23) for (BL-PS), and 3.5 (SD=5.51) for (FU-EOT), with unpaired t-test p-value of 0.005. Differences values for BL-PS, EOT-BL, FU-BL, and FU-EOT by dose group and genotype, are reported in Table 9. The Practice Effect (PE), BL-PS, for the 3 mg dose group was −3.5 (SD=4.57) different compared to PE of −0.21 (SD=4.23) for all subjects. The other dose groups PE changes were closer to the overall value for all subjects.

TABLE 5
Changes in DSST Scores Dose and Genotype

| | | | | | |
|---|---|---|---|---|---|
| Result | Stats | 3 mg | 10 mg | 30 mg | 90 mg |

BL-PS (Practice Effect)

| | | | | | |
|---|---|---|---|---|---|
| All | Ave (SD)^{N} | −3.50(4.57)⁸ | 1.22(2.68)⁹ | 1.44(2.96)⁹ | −0.38(5.18)⁸ |
| E4 | Ave (SD)^{N} | −3.00(4.36)³ | 1.29(3.04)⁷ | 3.50(2.89)⁴ | −1.67(8.50)³ |
| E3 | Ave (SD)^{N} | −3.80(5.17)⁶ | 1.00(1.41)² | −0.20(1.92)⁵ | 0.40(2.97)⁵ |

EOT-BL

| | | | | | |
|---|---|---|---|---|---|
| All | Ave (SD)^{N} | 4.43(9.16)⁷ | 1.00(5.50)⁹ | 0.33(3.61)⁹ | 1.13(4.55)⁸ |
| E4 | Ave (SD)^{N} | 11.50(16.3)² | 0.71(6.29)⁷ | −1.00(2.94)⁴ | 1.33(7.09)³ |
| E3 | Ave (SD)^{N} | 1.60(4.98)⁶ | 2.00(1.41)² | 1.40(4.04)⁵ | 1.00(3.32)⁵ |

FU-BL

| | | | | | |
|---|---|---|---|---|---|
| All | Ave (SD)^{N} | 5.38(8.50)⁸ | 3.11(5.01)⁹ | 4.00(5.68)⁹ | 6.63(11.4)⁸ |
| E4 | Ave (SD)^{N} | 6.67(12.4)³ | 3.86(5.52)⁷ | 0.25(3.30)⁴ | 15.00(15.1)³ |
| E3 | Ave (SD)^{N} | 4.60(6.88)⁶ | 0.50(0.71)² | 7.00(5.57)⁵ | 1.60(5.55)⁵ |

FU-EOT

| | | | | | |
|---|---|---|---|---|---|
| All | Ave (SD)^{N} | 1.86(4.91)⁸ | 2.11(3.95)⁹ | 3.67(4.30)⁹ | 5.50(8.35)⁸ |
| E4 | Ave (SD)^{N} | −1.00(1.41)³ | 3.14(3.80)⁷ | 1.25(4.99)⁴ | 13.67(8.50)³ |
| E3 | Ave (SD)^{N} | 3.00(5.48)⁶ | −1.50(2.12)² | 5.60(2.79)⁵ | 0.60(2.41)⁵ |

The EOT-BL values did not show dose dependency and were generally similar to EOT-PS values except for the 3 mg group, where EOT-BL was 4.43 (SD=9.16) and EOT-PS was 0.57 (SD=7.14). All four dose groups had positive values for EOT-BL and EOT-PS. The FU-BL values from 10 mg to 90 mg increase with dose as did the FU-PS measure for all four doses, giving a modest dose dependent trend (R²=0.65). All FU-BL DSST change values are positive and larger than the EOT-BL values (Table 9). For FU-EOT a dose related trend (R²=0.957) is observed from 3 mg to 90 mg. None of the FU-EOT dose group values are significantly different from each other, from dose group BL-PS values, or from the overall BL-PS value. The 10 mg group FU-PS (3.14, SD=3.80, n=5) was close to SS different from the BL-PS value for all subjects (0.21, SD=4.23, n=34) with an unpaired t-test p value of 0.066. The 10, 30 and 90 mg dose groups were all similar in that a small positive change was observed (EOT-BL, followed by a larger FU-EOT value to give positive (3.11 to 6.63) FU-BL values. The 3 mg group diverged from this only because of the unexpected large (−3.57) decrease from PS to BL.

Splitting dose groups by ApoE4 positive (E4), or ApoE3 only (E3), genotypes provides some interesting observations. Both E3 and E4 genotypes in the 3 mg group showed an aberrant PE effect. For the 30 mg group, several measured changes achieved SS when divided out by genotype. The FU-BL change for the E3s (7.00, SD=5.57, n=5) had p value of 0.042 compared to the E3 BL-PS (−0.20, SD=1.98, n=5), and p=0.013 when compared to BL-PS for all subjects. The FU-EOT for the 30 mg E3s (5.60, SD=2.79) was SS different from E3 BL-PS (p=0.006) and from all subject BL-PS (p=0.005). The 30 mg E4s (n=4) did not show SS differences, but the 90 mg E4s (n=3) did despite the small number of subjects. FU-EOT (13.67, SD=8.5, n=3) was for the 90 mg E4s, which is SS different from BL-PS for all subjects (p=0.01).

Reference is made to Chamberlain et al., An Exploratory Phase IIa Study of the PPAR delta/gamma Agonist T3D-959 Assessing Metabolic and Cognitive Function in Subjects with Mild to Moderate Alzheimer's Disease, Journal of Alzheimer's Disease, 73, 1-19, 2019, herein incorporated by reference in its entirety.

### Example 22

FDG-PET Data from Clinical Study with Compound of Example 11

A total of 36 subjects were enrolled into a two week-long treatment study which included FDG-PET (¹⁸Fluorodexoyglucose-Positron Emission Tomography) scans. The average age was 75 years with more than half of the subjects ranging between ages 65 to 84. Males and females were equally represented across all dose levels. One half of the enrolled subjects carried one or two copies of the E4 allele for APOE genotype. FDG-PET scans were obtained at three clinical sites. PET scans were obtained at baseline (BL) and again at end of treatment (EOT) for patients in all four dosage groups of Compound of Example 11 (3 mg, 10 mg, 30 mg, and 90 mg). The imaging protocol developed for the AD Neuroimaging Initiative (ADNI2) was used to collect the data. Overnight fasted subjects (blood glucose <180 mg/dL) received IV injection of 5mCi [F¹⁸] fluoro-deoxyglucose as a single bolus. Subjects were instructed to lay supine with eyes open and forward. Thirty minutes after dosing, six 5-minute (total 30-min) emission scans were acquired. FDG-PET measurements obtained are relative to two reference regions, Whole Brain (WB) and cerebral White Matter (WM), for the computation of the changes in Relative Cerebral Metabolic Rate for glucose over the dosing period or: ΔR CMRgl (EOT-BL). The key primary outcome, was a global index, (sROI index) calculated from the average bq/voxel reading over an empirically pre-specified statistical Region of Interest (sROI), known to be affected by AD, which is normalized by the average bq/voxel for an empirically pre-specified statistic ROI that is relatively spared. Change in the sROI index from BL to EOT is reported as A sROI. A second outcome was the determination of A R CMRgl (EOT-BL) for four pre-specified known AD-affected regions of interest (ROIs): 1) Posterior Cingulate (PC), 2) Precuneus (PreC), 3) Bilateral Middle Temporal Gyrus (BMTG), and 4) Right Inferior Parietal Lobule (RIPL). The final main outcome was an exploratory voxel-wise analysis of the whole brain to identify Regions of Statistically Significant Differences (ROSD) for A R CMRgl (EOT-BL) with uncorrected p<0.005. The voxel-wise analysis results are presented as slice by slice statistical map display superimposed on the anatomical images with ROSDs highlighted in yellow. The R CMRgl values referred to in this report are calculated as the ratio of the average of the bq/voxel reading for each voxel, over each ROI, and divided by the average bq/voxel over the reference region used, e.g., WB or WM.

Increases and decreases in relative regional glucose metabolism (A R CMRgl (EOT-BL)) were observed over the treatment period. Table 6 below shows multiple regions of the brain with positive A R CMRgl (EOT-BL) Relative to Average Whole Brain for combined dose groups. These are regions that are demonstrated to be responding better to drug than the average whole brain.

TABLE 6
Brain Regions with Positive Changes in Relative
CMRgI (EOT-BL) Relative to average Whole Brain

| Brain Regions | □ R CMRgI (EOT-BL) | P-value |
|---|---|---|
| Orbital front intersection L | 0.03 ± 0.04 | 3.0E−05 |
| Orbital front intersection R | 0.03 ± 0.03 | 3.0E−05 |
| Insula intersection L | 0.03 ± 0.03 | 1.0E−6 |
| Insula L | 0.02 ± 0.03 | 1.1E−04 |
| Insula intersection R | 0.03 ± 0.04 | 2.0E−05 |
| Insula R | 0.02 ± 0.04 | 1.3E−03 |
| Cingulum Ant intersection L | 0.04 ± 0.05 | 1.3E−04 |
| Cingulum Ant L | 0.03 ± 0.04 | 5.2E−04 |
| Cingulum Ant intersection R | 0.03 ± 0.04 | 1.9E−04 |
| Cingulum Ant R | 0.02 ± 0.05 | 7.9E−03 |
| Putamen intersection L | 0.06 ± 0.06 | 1.0E−05 |
| Putamen L | 0.05 ± 0.07 | 5.0E−05 |
| Putamen intersection R | 0.06 ± 0.06 | 1.0E−05 |
| Putamen R | 0.05 ± 0.06 | 3.0E−05 |

The effect of drug on the FDG-PET outcomes appears to be dose-dependent, with larger effects observed at larger doses. This observation comes from the sROI, and anatomical ROI analyses as well as the exploratory voxel-wise SPM analysis. The image displays below from the voxel-wise analysis shows a clear increase in the spatial extent of the regions of the yellow regions from 70 voxels to 2136 voxels as the dose increases from 10 mg to 90 mg. The yellow regions are made up of voxels with statistically significant differences from baseline to end of treatment (ROSDs).

ROSDs with Positive a R CMRgl (EOT-BL) Relative to Whole Brain (p<0.005) for with Increasing Dose

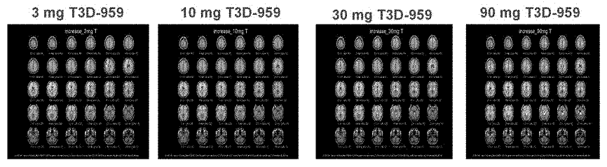

FIG. 1 illustrates comparisons of slide image displays of regions of statistically significant differences (ROSDs) with positive R CMRgl. Reference is made to Chamberlain et al., An Exploratory Phase IIa Study of the PPAR delta/gamma Agonist T3D-959 Assessing Metabolic and Cognitive Function in Subjects with Mild to Moderate Alzheimer's Disease, Journal of Alzheimer's Disease, 73, 1-19, 2019, herein incorporated by reference in its entirety.

### Example 23

Plasma Metabolomic Data from Clinical Study with Compound of Example 11

Fasted plasma metabolomics biomarkers were examined seeking evidence of systemic and brain pharmacological effects of Compound from Example 11. Over 800 chemically defined metabolites were examined for each dose group and a ratio EOT/BL) was calculated with an associated p value. Four dose groups were tested, 3, 10 30 and 90 mg. In general, the 30 and 90 mg dose groups had the largest impact on the metabolomics profile, each with 120 metabolites with p<0.05, while the 3 and 10 mg groups had smaller effects (40 and 61 with p<0.05 respectively). The metabolites were split into about 60 families. Most of these showed little change with treatment but several had consistent and significant changes. All three branched chain amino acids (BCAA), Leu, Ile and Val are significantly decreased (p<0.05) in the 90 mg group. BCAAs are positively correlated with insulin resistance and diabetes. Supporting this observation, several key products of BCAA catabolism in the form of acyl carnitines, are similarly decreased in treatment in the 90 mg group (Table 5). Some of these metabolites, such as isovaleryl and isobutyryl carnitine are part of a principal component shown to be positively associated with insulin resistance. The numbers in Table 7 are ratios of the dose group averages end of treatment (EOT) to baseline (BL). A green box indicates a statistically significant (p<0.05) decrease in metabolite. The light green indicates the p value is between 0.05 and 0.1. Statistical comparisons between doses and visits were conducted using Two-Way Repeated Measure ANOVA. Reference is made to Newgard CB, 2017. Metabolomics and Metabolic Disease, Where Do We Stand? Cell Metabolism 25: 43-56, which is incorporated by reference with regard to the example.

TABLE 7
Clinical Changes in Branched Chain Amino Acids

| Metabolite | 3 mg | 10 mg | 30 mg | 90 mg |
|---|---|---|---|---|
| leucine | 0.95 | 0.98 | 1 | 0.8 |
| N-acetylleucine | 0.98 | 1 | 0.91 | 0.82 |
| isovalerylcarnitine (C5) | 1.11 | 0.97 | 0.99 | 0.68 |
| isoleucine | 0.93 | 0.94 | 0.96 | 0.84 |
| 2-methylbutyrylcarnitine (C5) | 0.99 | 1.02 | 0.96 | 0.81 |
| valine | 0.93 | 1.06 | 0.96 | 0.79 |
| isobutyrylcarnitine (C4) | 0.95 | 1.01 | 0.77 | 0.57 |

A limited number of ceramides/N-acyl sphingosines were included in the exploratory metabolomic analysis. Compound of Example 11 decreased the levels of several ceramides, including N-palmitoyl and N-stearoyl sphingosine at the higher doses, as shown in Table 8. Ceramides are postulated to be mediators of insulin resistance and metabolic disease. Recent reports suggest strong association of specific ceramide species (e.g. C16:0, or N-palmitoyl-shingosine) with metabolic diseases. Reference is made to Turpin, S M, Nicholls H T, Willmes D M, Mourier An, Brodesser S, Wunderlich C M, Mauer J, Xu E, Hammerschmidt P, Bronneke H S, Trifunovic A, LoSassao G, Wunderlich F T, Kornfeld J-W, Bluher M, Kronke M, Bruning J C. Obesity-induced CerS6-dependent C16:0 ceramide production promotes weight gain and glucose intolerance. Cell Metabolism 20: 678-686, 2014, which is incorporated by reference with regard to the example.

TABLE 8
Clinical bserved Changes in Ceramides

| Ceramides | 3 mg | 10 mg | 30 mg | 90 mg |
|---|---|---|---|---|
| ceramide (d16:1/24:1, d18:1/22:1) | 1.13 | .095 | 0.71 | 0.84 |
| ceramide (d18:1/14:0, d16:1/16:0) | 1.1 | 1.05 | 0.86 | 0.77 |
| ceramide (d18:1/17:0, d17:1/18:0) | 1.03 | 1.12 | 0.91 | 0.84 |
| ceramide (d18:1/20:0, d16:1/22:0 | 1.12 | 1.05 | 0.83 | 0.84 |
| d20:1/18:0) | | | | |
| N-palmitoyl-sphingosince | 1.06 | 1.02 | 0.89 | 0.89 |
| (d18:1/16:0) | | | | |
| N-stearoyl-sphinogosince | 1.05 | 0.98 | 0.82 | 0.82 |
| (d18:1/18:0) | | | | |
| Ceramide (d18:2/24:1, d18:1/24:2) | 1.08 | 0.96 | 0.92 | 0.99 |

The overall profile from the metabolomic data suggests that the two higher doses increased fatty acid oxidation. Over 30 acyl carnitine species were measured in this metabolomic analysis. The 30 and 90 mg doses increased a wide array of these fatty acid-derived acylcarnitine species, ranging from the end product C2 (acetyl) carnitine through the even-chain medium (C4 to C12) and long chain (C14-C22) species, Table 9.

TABLE 9
Clinically Increase Levels of Acyl Carnitines

| Acyl Carnitines | 3 mg | 10 mg | 30 mg | 90 mg |
|---|---|---|---|---|
| acetylcarnitine (C2) | 1.14 | 1.03 | 1.19 | 1.23 |
| 3-hydroxybutyrlcarnite | 1.2 | 1.19 | 1.26 | 1.53 |
| octanoylcarnitine (C8) | 1.33 | 1.21 | 1.44 | 1.58 |
| laurylcarnitine (C12) | 1.4 | 1.17 | 1.35 | 1.56 |
| Myristoycarnitine (C14) | 1.32 | 1.13 | 1.29 | 1.56 |
| Palmitoylcaritine (C16) | 1.25 | 1.12 | 1.29 | 1.31 |
| Palmitoleoylcarnitine (C16:1)* | 1.44 | 1.11 | 1.66 | 1.79 |
| Stearoylcarnitine (C18) | 1.11 | 1.19 | 1.24 | 1.18 |
| Linoleoylcarnitine (C18:2)* | 1.22 | 1.21 | 1.37 | 1.22 |
| Linolenoylcarnitine (C18:3)* | 1.17 | 1.19 | 1.35 | 1.25 |
| Oleoylcarnitine (C18:1) | 1.34 | 1.1 | 1.51 | 1.46 |
| Arachidoylcarnitine (C20)* | 1.11 | 1.09 | 1.16 | 1.17 |
| Arachidonoylcarnitine (C20:4) | 1.34 | 1.23 | 1.34 | 1.31 |
| Adrenoylcarnitine (C22:4)* | 1.41 | 1.23 | 1.44 | 1.54 |
| Dihomo-linoleoylcarnitine (C20:2)* | 1.24 | 1.24 | 1.47 | 1.54 |
| Eicosenoylcarnitine (C20:1) | 1.22 | 1.03 | 1.43 | 1.61 |
| Docosapentaenoylcarnitine | 1.39 | 1.18 | 1.65 | 1.45 |
| (C22:5n3)* | | | | |
| Margaroylcarnitine* | 1.03 | 1.15 | 1.14 | 1.21 |

This profile is consistent with increased flux of fatty acids into the beta-oxidation pathway. The red highlighted entries in Table 8 indicate a statistically significant (p<0.05) increase in the ratio (EOT/BL) for a metabolite, while pink indicates a p value between 0.05 and 0.1. Reference is made to Newgard CB, 2017. Metabolomics and Metabolic Disease, Where Do We Stand? Cell Metabolism 25: 43-56, which is incorporated by reference with regard to the example.

Reference is made to Chamberlain et al., An Exploratory Phase IIa Study of the PPAR delta/gamma Agonist T3D-959 Assessing Metabolic and Cognitive Function in Subjects with Mild to Moderate Alzheimer's Disease, Journal of Alzheimer's Disease, 73, 1-19, 2019, herein incorporated by reference in its entirety.

Those skilled in the art to which the present disclosure pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present disclosure is, therefore, indicated by the appended claims rather than by the foregoing description or drawings. Consequently, while the present invention has been described with reference to particular embodiments, modifications of structure, sequence, materials and the like apparent to those skilled in the art still fall within the scope as claimed.

1. A method for treating a subject having a betacoronavirus infection comprising administering a therapeutically effective amount of a Peroxisome Proliferator-Activated Receptor (PPAR) agonist, which penetrates the blood brain barrier (BBB).

2. The method of claim 1, wherein the betacoronovirus is selected from one or more of SARS-CoV-2, SARS-CoV-1, MERS-CoV, NCoV-OC43, HCoV-HKU1, and a novel beta coronavirus.

3. The method of claim 1, wherein the beta coronavirus infection causes or exacerbates one or more of Acute Respiratory Distress Syndrome (ARDS), Cytokine Release Syndrome (CRS), a central nervous system disorder, delirium, cognitive impairment, cardiovascular disease, kidney disease, intestinal disease, liver disease, Deep Vein Thrombosis (DVT), and elevated blood glucose levels.

4. The method of claim 1, wherein the therapeutically effective amount provides pharmacologically useful concentrations in the brain.

5. The method of claim 1, wherein the PPAR agonist is a PPAR-delta agonist, a PPAR-gamma agonist, or a dual PPAR delta and gamma agonist.

6. The method of claim 5, wherein the PPAR agonist is a compound of Formula I: 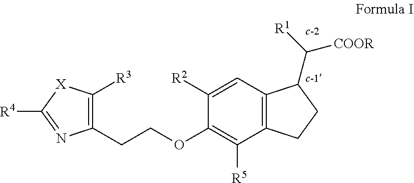
R is H or C₁-C₆ alkyl;
R¹ is H, COOR, C₃-C₈ cycloalkyl, or C₁-C₆ alkyl, C₂-C₆ alkenyl, or C₁-C₆ alkoxy, each of which may be unsubstituted or substituted with fluoro, methylenedioxyphenyl, or phenyl which may be unsubstituted or substituted with R⁶;
R² is
(i) H, halo, or C₁-C₆ alkyl, which may be unsubstituted or substituted with C₁-C₆ alkoxy, oxo, or fluoro; or
(ii) phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl, each of which may be unsubstituted or substituted with R⁶;
R³ is H, C₁-C₆ alkyl, or phenyl which may be unsubstituted or substituted with R⁶;
X is O or S;
R⁴ is
(i) C₁-C₆ alkyl or C₃-C₈ cycloalkyl,
a. either of which may be unsubstituted or substituted with fluoro, oxo, or C₁-C₆ alkoxy, which may be unsubstituted or substituted with C₁-C₆ alkoxy or phenyl optionally substituted with R⁶,
or
b. either of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1, 4-benzodioxanyl, each of which may be unsubstituted or substituted with R⁶,
or
c. C₁-C₆ alkyl may also be substituted with
i. C₃-C₈ cycloalkyl;
ii. phenoxy which may be unsubstituted or substituted with R⁶; or
iii. phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1, 4-benzodioxanyl, each of which may be unsubstituted or substituted with R⁶, or
(ii) phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl, each of which may be unsubstituted or substituted with R⁶ or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy, each of which may be unsubstituted or substituted with R⁶;
R⁵ is H, halo, or C₁-C₆ alkyl optionally substituted with oxo;
R⁶ is halo, CF₃, C₁-C₆ alkyl optionally substituted with oxo or hydroxy, or C₁-C₆ alkoxy optionally substituted with fluoro;
or a pharmaceutically acceptable salt or ester thereof.

7. The method of claim 6, wherein
R¹ is H or C₁-C₆ alkyl;
R² is H or halo;
R³ is C₁-C₆ alkyl;
R⁴ is unsubstituted phenyl or phenyl substituted with one or more halogen, C₁-C₆ alkyl, C₁-C₆ haloalkyl, or C₁-C₆ alkoxy;
R⁵ is H or halo; and
X is O or S.

8. The method of claim 6, wherein the designated c-1′ has S relative stereochemistry.

9. The method of claim 6, wherein the PPAR agonist is selected from: 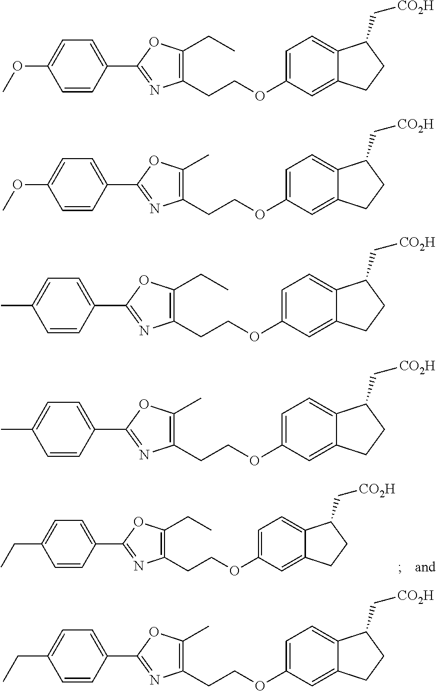
or a pharmaceutically acceptable salt or ester thereof.

10. The method of claim 6, wherein the pharmaceutically acceptable salt is selected from the group consisting of alkali metal salts, alkaline earth metal salts, ammonium salts with organic bases, and basic nitrogen containing groups in the conjugate base that is quaternized with agents selected from the group consisting of alkyl halides and aralkyl halides, or other alkylating agents.

11. The method according to claim 10 wherein salt is a potassium, sodium, calcium, magnesium, lysine, choline or meglumine salt thereof.

12. The method of claim 1, wherein the PPAR agonist is (1S)-1H-Indene-1-acetic acid, 5-[2-[5-ethyl-2-(4-methoxyphenyl)-4-oxazolyl]ethoxy]-2,3-dihydro-, sodium salt (1:1)

13. The method of claim 2, wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat Acute Respiratory Distress Syndrome (ARDS) associated with COVID-19 disease.

14. The method of claim 2, wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat a central nervous system disorder, delirium, or cognitive impairment associated with COVID-19 disease.

15. The method of claim 2, wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat cardiovascular disease associated with COVID-19 disease.

16. The method of claim 2, wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat kidney disease associated with COVID-19 disease.

17. The method of claim 2, wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat cardiovascular disease associated with COVID-19 disease.

18. The method of claim 2, wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat intestinal disease associated with COVID-19 disease.

19. The method of claim 2, wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat liver disease associated with COVID-19 disease.

20. The method of claim 2, wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat deep vein thrombosis associated with COVID-19 disease.

21. The method of claim 2, wherein (1S)-1H-Indene-1-acetic acid, 5-[2-[5-ethyl-2-(4-methoxyphenyl)-4-oxazolyl]ethoxy]-2,3-dihydro-, sodium salt (1:1) is used to treat elevated blood glucose levels associated with COVID-19 disease.

22. The method of claim 1, wherein the PPAR agonist is administered intravenously, orally, buccally, transdermally, rectally, nasally, optically, intrathecally, or intra-cranially

23. The method of claim 1, further comprising administration of one or more additional therapeutic agents.

24. The method according to claim 23, wherein one or more additional therapeutic agent is used to treat COVID-19.

25. The method according to claim 24, wherein the one or more additional therapeutic agent is an antiviral.

26. The method according to claim 24, wherein one or more additional therapeutic agents is remdesivir or nafamostat mesylate.

27. The method according to claim 24 one or more of remdesivir, niclosamide, favipiravir (favilavir, Avigan), nafamostat, camostat, galidesivir, Jakafi (ruxolitinib), losartan, other angiotensin II receptor antagonist, and tocilizumab.

28. The method of claim 1, wherein the PPAR agonist is characterized by a rat brain to plasma ratio of greater than about 20%, about 12 hours after oral dosing.
